(19) United States
(12) Patent Application Publication
(10) Pub. No.: US 2026/0325846 A1
(43) Pub. Date: Oct. 22, 2026

(54) DAYTIME STIMULATION THERAPY FOR DISORDERED BREATHING

(71) Applicant: ZOLL Medical Corporation, Chelmsford, MA (US)

(72) Inventors: Robin E. Germany, Dayton, MN (US); Kristofer J. James, Eagan, MN (US); Randy W. Westlund, St. Paul, MN (US); Gary A. Freeman, Waltham, MA (US)

(73) Assignee: ZOLL Medical Corporation, Chelmsford, MA (US)

(21) Appl. No.: 19/467,973

(22) Filed: Feb. 3, 2026

Related U.S. Application Data
(60) Provisional application No. 63/760,290, filed on Feb. 19, 2025.

(51) Int. Cl. A61N 1/36; A61B 5/08; A61N 1/372

(52) U.S. Cl. CPC A61N 1/3611; A61B 5/0816; A61N 1/36139; A61N 1/36167; A61N 1/37211

(57) ABSTRACT

Examples of the disclosure include an implantable pulse generator (IPG) for treating Cheyne-Stokes respiration (CSR) in an awake patient, the IPG configured to provide electrical stimulation to a target tissue comprising a phrenic nerve via at least one implantable stimulation lead, the IPG comprising at least one processor, at least one memory, and stimulation circuitry, wherein the at least one processor is configured to enter a non-sleep time therapy operational mode during a pre-determined non-sleep time period for a patient, monitor a signal from at least one respiration sensor coupled to the IPG, identify patient CSR based at least in part on a presence of a CSR pattern in the signal, and control the stimulation circuitry to provide a first delivery of the electrical stimulation to the phrenic nerve for a pre-programmed time period in response to the determination of the presence of the CSR pattern in the monitored signal.

### CROSS-REFERENCE TO RELATED APPLICATIONS

This application claims priority under 35 U.S.C. § 119(e) to U.S. Provisional Patent Application Ser. No. 63/760,290 titled “DAYTIME STIMULATION THERAPY FOR DISORDERED BREATHING,” filed Feb. 19, 2025, the entire contents of which is incorporated herein by reference for all purposes.

### TECHNICAL FIELD

At least one example in accordance with the present disclosure relates generally to breathing disorders.

### BACKGROUND

Breathing that exhibits various characteristics that deviate from normal respiration and that may be indicative of unhealthy respiration may be described as disordered breathing. Periodic breathing refers to abnormal respiration patterns, or disordered breathing patterns, that alternate between hypopnea (i.e. diminished breathing) and hyperpnea (i.e. fast, deep breathing.) One form of periodic breathing is Cheyne-Stokes Respiration (CSR), which can occur in patients with congestive heart failure (CHF). Briefly, CSR arises principally due to a time lag between blood carbon dioxide (CO₂) levels sensed by the central nervous system and the blood CO₂ levels. With CHF, poor cardiac function results in poor blood flow to the brain such that the central nervous system responds to blood CO₂ levels that are no longer properly representative of the overall blood CO₂ levels in the body. Hence, the central nervous system triggers an increase in the depth and frequency of breathing in an attempt to compensate for perceived high blood CO₂ levels even though the blood CO₂ levels have already dropped. By the time the central nervous system detects the drop in blood CO₂ levels and slows respiration in response, the blood CO₂ levels have already increased. This cycle becomes increasingly unbalanced until respiration periodically alternates between hypopnea (or hypoventilation) and hyperpnea (or hyperventilation). The wildly fluctuating blood chemistry levels can exacerbate CHF and other medical conditions. Moreover, the periods of hypopnea can be sufficiently pronounced that apnea episodes occur.

Sleep disordered breathing exists in several forms, including central sleep apnea (CSA), obstructive sleep apnea (OSA), a combination disorder, which may include mixed apnea events, and other types of breathing disorders. CSA includes apnea events caused by a temporary lack of respiratory drive. With CSA, the brain does not properly signal the phrenic nerves to contract the diaphragm, pulling air into the lungs. OSA includes apnea events resulting from a partial or complete obstruction of the airway resulting in a reduction in airflow relative to airflow without the obstruction. This decrease in airway patency may be due to a person's anatomy or by relaxation of chest and neck muscles during sleep. These muscles may include the tongue. In the combination disorder, mechanisms of CSA and OSA are both present in a patient.

### SUMMARY

Examples of the methods and systems discussed herein are not limited in application to the details of construction and the arrangement of components set forth in the following description or illustrated in the accompanying drawings. The methods and systems may be capable of implementation in other embodiments and of being practiced or of being carried out in various ways. Examples of specific implementations are provided herein for illustrative purposes and are not intended to be limiting. Acts, components, elements, and features discussed in connection with any one or more examples may be configured to operate and/or be implemented in a similar role in any other examples.

The phraseology and terminology used herein is for the purpose of description. References to examples, embodiments, components, elements, or acts of the systems and methods herein referred to in the singular may also embrace embodiments including a plurality. Similarly, references in plural to embodiments, components, elements, or acts may be implemented as a singularity. References in the singular or plural form may therefore not be intended to limit the presently disclosed systems or methods, their components, acts, or elements. The use herein of “including,” “comprising,” “having,” “containing,” “involving,” and variations so forth, may encompass the items listed thereafter and equivalents thereof as well as additional items.

References to “or” may be construed as inclusive so that any terms described using “or” may indicate any of a single, more than one, and all of the described terms. For example, the phrase “at least one of A or B” may refer A and/or B—that is, A only, B only, or A and B together. In addition, in the event of inconsistent usages of terms between this document and documents incorporated herein by reference, the term usage in the incorporated documents is supplementary to this document. For irreconcilable differences, the term usage in this document controls.

According to at least one aspect of the present disclosure, an implantable pulse generator (IPG) for treating Cheyne-Stokes respiration (CSR) in an awake patient is provided, the IPG configured to provide electrical stimulation to a target tissue comprising a phrenic nerve via at least one implantable stimulation lead configured to couple to the IPG, the IPG comprising: at least one processor, at least one memory, and stimulation circuitry; and wherein the at least one processor is configured to enter a non-sleep time therapy operational mode during a pre-determined non-sleep time period for a patient, monitor a signal from at least one respiration sensor coupled to the IPG, identify CSR of the patient based at least in part on a determination of a presence of a CSR pattern in the monitored signal, and control the stimulation circuitry to provide a first delivery of the electrical stimulation to the phrenic nerve for a pre-programmed time period in response to the determination of the presence of the CSR pattern in the monitored signal.

In at least one example, the at least one processor is configured to control the stimulation circuitry to reduce an energy of the electrical stimulation at a conclusion of the pre-programmed time period, redetermine the presence of the CSR pattern in the monitored signal after the reduction in the electrical stimulation energy, and if the redetermination indicates the presence of the CSR pattern in the monitored signal, then control the stimulation circuitry to provide a second delivery of the electrical stimulation to the phrenic nerve for the pre-programmed time period, else discontinue the electrical stimulation. In at least one example, the at least one processor is configured to adjust at least one electrical stimulation parameter for the second delivery responsive to the redetermination of the presence of the CSR pattern.

In at least one example, the at least one processor is configured to reduce the energy of the electrical stimulation to zero at the conclusion of the pre-programmed time period. In at least one example, the at least one processor is configured to reduce the energy of the electrical stimulation at the conclusion of the pre-programmed time period from a first value corresponding to the first delivery of the electrical stimulation to a second value, the second value being less than the first value and greater than zero. In at least one example, the at least one processor is configured to discontinue delivery of the electrical stimulation at a conclusion of a pre-determined CSR therapy duration time period.

In at least one example, the at least one processor is further configured to monitor a signal from at least one patient motion sensor, and discontinue the electrical stimulation responsive to the signal from the at least one patient motion sensor indicative of patient movement above a patient movement threshold. In at least one example, the IPG comprises a wireless communications interface. In at least one example, the at least one processor is further configured to store a CSR event marker in the at least one memory, and transmit the CSR event marker to an external computing device via the wireless communications interface. In at least one example, the wireless communications interface is configured to communicate via one or more of telemetry or a Bluetooth® communications protocol.

In at least one example, the wireless communications interface is configured to communicate with at least one patient-specific external computing device. In at least one example, the at least one patient-specific external computing device comprises one or more of a smartphone, a smartwatch, a laptop, or a wearable computing device. In at least one example, the at least one processor is further configured to discontinue the electrical stimulation responsive to reception, via the wireless communications interface, of a first user input to the at least one patient-specific external computing device to stop the electrical stimulation. In at least one example, the at least one processor is further configured to automatically resume providing the electrical stimulation after a pre-determined period of time elapses after reception of the first user input to stop the electrical stimulation.

In at least one example, the at least one processor is further configured to resume providing the electrical stimulation responsive to reception of a second user input to the at least one patient-specific external computing device to resume the electrical stimulation. In at least one example, the IPG is further configured to provide an alert to the at least one patient-specific external computing device indicative of an initiation of the electrical stimulation prior to delivery of the electrical stimulation. In at least one example, the at least one processor is further configured to automatically commence delivery of the electrical stimulation a pre-determined amount of time after providing the alert. In at least one example, the at least one processor is configured to enter the non-sleep time therapy operational mode during the pre-determined non-sleep time period for the patient responsive to a user input therapy commencement instruction to the at least one patient-specific external computing device.

In at least one example, the IPG is further configured to output an alarm indicative of the CSR pattern determination to the at least one patient-specific external computing device. In at least one example, the at least one processor is configured to monitor a signal from at least one patient fluid load sensor coupled to the IPG, the signal being indicative of a fluid load of the patient, and adjust an energy of the electrical stimulation based on the signal from the at least one patient fluid load sensor. In at least one example, the identification of CSR of the patient comprises implementation of a detection algorithm to determine the presence of the CSR pattern based on the monitored signal.

In at least one example, the at least one processor is configured to implement the detection algorithm. In at least one example, the IPG is configured to communicatively couple to at least one external computing device configured to implement the detection algorithm, provide at least one of the monitored signal or information derived from the signal from the monitored signal to the at least one external computing device, and receive a CSR pattern detection indication from the at least one external computing device, wherein the determination of the presence of the CSR pattern in the monitored signal is based on the CSR pattern detection indication. In at least one example, the at least one processor is configured to monitor the signal from the at least one respiration sensor for at least a threshold duration, and the detection algorithm is configured to determine that the CSR pattern is present for at least the threshold duration.

In at least one example, the threshold duration comprises one or more complete cycles of the CSR pattern. In at least one example, the threshold duration comprises one or more of two complete cycles of the CSR pattern or 30-120 seconds. In at least one example, the detection algorithm is further configured to determine a correlation index between signal morphology elements of the monitored signal and identify the signal morphology elements as CSR cycles based on the correlation index. In at least one example, the at least one processor is configured to receive a sensor signal indicative of patient activity from one or more of an implanted accelerometer or an accelerometer coupled to an exterior surface of the patient, and the determination of the presence of the CSR pattern in the monitored signal is based at least in part on the patient activity.

In at least one example, the at least one processor is configured to receive a sensor signal indicative of a patient heart rate from one or more of an internal heart rate sensor or an external heart rate sensor, and wherein the determination of the presence of the CSR pattern in the monitored signal is based at least in part on the patient heart rate. In at least one example, the at least one processor is configured to increase an energy of the electrical stimulation from an initial energy level up to a maximum energy level over the pre-programmed time period or over one or more successive stimulation delivery time periods. In at least one example, the at least one processor is configured to enter the non-sleep time period therapy operational mode based at least in part on a pre-programmed start time-of-day and a pre-programmed end time-of-day.

In at least one example, the at least one processor is configured to monitor a signal from at least one patient motion sensor indicative of one or more of patient movement or patient position, determine one or more of motion or position parameters based on the signal, and enter the non-sleep time period therapy operational mode based at least in part on the one or more of motion or position parameters. In at least one example, the at least one patient motion sensor comprises at least one of an implanted accelerometer or an accelerometer coupled to an exterior surface of the patient. In at least one example, the at least one processor is further configured to enter a sleep apnea therapy operational mode during a pre-determined sleep time period for the patient identified at least in part based on the pre-programmed start time-of-day and the pre-programmed end time-of-day.

In at least one example, the at least one processor is further configured to control the stimulation circuitry to deliver electrical stimulation during the pre-determined sleep time period at a higher energy than during the pre-determined non-sleep time period. In at least one example, the IPG comprises a hermetically sealed casing, and wherein the at least one processor, the at least one memory, and the stimulation circuitry are disposed within the hermetically sealed casing. In at least one example, the IPG comprises at least one battery disposed within the hermetically sealed casing, and wherein the at least one battery comprises one or more of a rechargeable battery or a non-rechargeable battery.

In at least one example, the at least one battery comprises the rechargeable battery and the IPG comprises magnetic inductive charging circuitry configured to provide a recharging current to the rechargeable battery. In at least one example, the IPG comprises a header coupled to the hermetically sealed casing, the header comprising one or more receptacles configured to couple to the at least one implantable stimulation lead configured to provide electrical stimulation to the phrenic nerve. In at least one example, the target tissue comprises an upper-airway nerve and the header comprises one or more receptacles configured to couple to at least one implantable stimulation lead configured to provide electrical stimulation to the upper-airway nerve.

In at least one example, the one or more receptacles are configured to couple to the at least one implantable stimulation lead comprising a cuff electrode or a transvenous electrode. In at least one example, the one or more receptacles are configured to couple to the at least one implantable stimulation lead comprising the at least one respiration sensor. In at least one example, the header comprises at least one receptacle configured to couple to at least one implantable sensing lead comprising the at least one respiration sensor. In at least one example, the IPG is configured to provide one or more of bilateral phrenic nerve stimulation or unilateral phrenic nerve stimulation. In at least one example, the at least one respiration sensor comprises a transthoracic-impedance sensor.

In at least one example, the at least one processor is further configured to control the stimulation circuitry based at least in part on signals from at least one of an electromyography sensor, a cardiac cycle sensor, an airflow sensor, an internal pressure sensor, a pulse oximeter, an effort belt, or a peripheral arterial tone sensor. In at least one example, the at least one processor is further configured to select a stimulation mode to treat the CSR in the awake patient, wherein the selected stimulation mode may be a same mode as or a different mode from a sleep disordered breathing stimulation mode. In at least one example, the selected stimulation mode is one of an asynchronous stimulation mode, a synchronous stimulation mode, or an apnea-triggered stimulation mode. In at least one example, the selected stimulation mode may be a same mode as the sleep disordered breathing stimulation mode but with at least one stimulation parameter that is different between the selected stimulation mode and the sleep disordered breathing stimulation mode.

According to at least one example of the disclosure, an implantable pulse generator (IPG) for treating Cheyne-Stokes respiration (CSR) in an awake patient is provided, the IPG configured to provide electrical stimulation to a target tissue comprising a phrenic nerve via at least one implantable stimulation lead configured to couple to the IPG, the IPG comprising: at least one processor, at least one memory, and stimulation circuitry; and wherein the at least one processor is configured to enter a non-sleep time therapy operational mode during a pre-determined non-sleep time period for a patient, monitor a signal from at least one patient sensor coupled to the IPG, identify a CSR-related condition of the patient based at least in part on the monitored signal, and control the stimulation circuitry to provide a first delivery of the electrical stimulation to the phrenic nerve for a pre-programmed time period in response to the identification of the CSR-related condition.

In at least one example, the at least one processor is configured to control the stimulation circuitry to reduce an energy of the electrical stimulation at a conclusion of the pre-programmed time period, evaluate a presence of the CSR-related condition in the monitored signal after the reduction in the electrical stimulation energy, and if the evaluation indicates the presence of the CSR-related condition in the monitored signal, then control the stimulation circuitry to provide a second delivery of the electrical stimulation to the phrenic nerve for the pre-programmed time period, else discontinue the electrical stimulation. In at least one example, the at least one processor is configured to adjust at least one electrical stimulation parameter for the second delivery responsive to the presence of the CSR-related condition.

In at least one example, the at least one processor is configured to reduce the energy of the electrical stimulation to zero at the conclusion of the pre-programmed time period. In at least one example, the at least one processor is configured to reduce the energy of the electrical stimulation at the conclusion of the pre-programmed time period from a first value corresponding to the first delivery of the electrical stimulation to a second value, the second value being less than the first value and greater than zero. In at least one example, the at least one processor is configured to discontinue delivery of the electrical stimulation at a conclusion of a pre-determined CSR therapy duration time period.

In at least one example, the at least one processor is further configured to monitor a signal from at least one patient motion sensor, and discontinue the electrical stimulation responsive to the signal from the at least one patient motion sensor indicative of patient movement above a patient movement threshold. In at least one example, the IPG includes a wireless communications interface. In at least one example, the at least one processor is further configured to store a CSR event marker in the at least one memory, and transmit the CSR event marker to an external computing device via the wireless communications interface In at least one example, the wireless communications interface is configured to communicate via one or more of telemetry or a Bluetooth® communications protocol.

In at least one example, the wireless communications interface is configured to communicate with at least one patient-specific external computing device. In at least one example, the at least one patient-specific external computing device comprises one or more of a smartphone, a smartwatch, a laptop, or a wearable computing device. In at least one example, the at least one processor is further configured to discontinue the electrical stimulation responsive to reception, via the wireless communications interface, of a first user input to the at least one patient-specific external computing device to stop the electrical stimulation. In at least one example, the at least one processor is further configured to automatically resume providing the electrical stimulation after a pre-determined period of time elapses after reception of the first user input to stop the electrical stimulation.

In at least one example, the at least one processor is further configured to resume providing the electrical stimulation responsive to reception of a second user input to the at least one patient-specific external computing device to resume the electrical stimulation. In at least one example, the IPG is further configured to provide an alert to the at least one patient-specific external computing device indicative of an initiation of the electrical stimulation prior to delivery of the electrical stimulation. In at least one example, the at least one processor is further configured to automatically commence delivery of the electrical stimulation a pre-determined amount of time after providing the alert. In at least one example, the at least one processor is configured to enter the non-sleep time therapy operational mode during the pre-determined non-sleep time period for the patient responsive to a user input therapy commencement instruction to the at least one patient-specific external computing device.

In at least one example, the IPG is further configured to output an alarm indicative of the CSR event marker to the at least one patient-specific external computing device. In at least one example, the at least one processor is configured to monitor a signal from at least one patient fluid load sensor coupled to the IPG, the signal being indicative of a fluid load of the patient, and adjust an energy of the electrical stimulation based on the signal from the at least one patient fluid load sensor. In at least one example, the at least one patient sensor comprises at least one respiration sensor. In at least one example, the at least one processor is configured to monitor a signal from the at least one respiration sensor, identify CSR of the patient based at least in part on a determination of a presence of a CSR pattern in the monitored signal, and control the stimulation circuitry to provide the first delivery of the electrical stimulation to the phrenic nerve for the pre-programmed time period in response to the identification of the CSR-related condition and the determination of the presence of the CSR pattern in the monitored signal.

In at least one example, the at least one processor is configured to control the stimulation circuitry to reduce an energy of the electrical stimulation at a conclusion of the pre-programmed time period, evaluate the presence of the CSR-related condition and redetermine the presence of the CSR pattern in the monitored signal after the reduction in the electrical stimulation energy, and if the evaluation indicates the presence of the CSR-related condition and the CSR pattern in the monitored signal, then control the stimulation circuitry to provide a second delivery of the electrical stimulation to the phrenic nerve for the pre-programmed time period, else discontinue the electrical stimulation.

In at least one example, the identification of the CSR of the patient comprises implementation of a detection algorithm to determine the presence of the CSR pattern based on the monitored signal. In at least one example, the at least one processor is configured to implement the detection algorithm. In at least one example, the IPG is configured to communicatively couple to at least one external computing device configured to implement the detection algorithm, provide at least one of the monitored signal or information derived from the signal from the monitored signal to the at least one external computing device, and receive a CSR pattern detection indication from the at least one external computing device, wherein the determination of the presence of the CSR pattern in the monitored signal is based on the CSR pattern detection indication.

In at least one example, the at least one processor is configured to monitor the signal from the at least one respiration sensor for at least a threshold duration, and wherein the detection algorithm is configured to determine that the CSR pattern is present for at least the threshold duration. In at least one example, the threshold duration comprises one or more complete cycles of the CSR pattern. In at least one example, the threshold duration comprises one or more of two complete cycles of the CSR pattern or 30-120 seconds. In at least one example, the detection algorithm is further configured to determine a correlation index between signal morphology elements of the monitored signal and identify the signal morphology elements as CSR cycles based on the correlation index.

In at least one example, the at least one processor is configured to receive a sensor signal indicative of patient activity from one or more of an implanted accelerometer or an accelerometer coupled to an exterior surface of the patient, and wherein the determination of the presence of the CSR pattern in the monitored signal is based at least in part on the patient activity. In at least one example, the at least one processor is configured to receive a sensor signal indicative of a patient heart rate from one or more of an internal heart rate sensor or an external heart rate sensor, and wherein the determination of the presence of the CSR pattern in the monitored signal is based at least in part on the patient heart rate. In at least one example, the IPG is configured to monitor a signal from the at least one respiration sensor, and identify the CSR-related condition as one or more of a reduction in respiration rate or a reduction in tidal volume.

In at least one example, the reduction in respiration rate is a reduction below a threshold respiration rate or a percentage drop in respiration rate. In at least one example, the threshold respiration rate is 12 breaths per minute. In at least one example, the percentage drop is 10-50%. In at least one example, the reduction in tidal volume is a reduction below a threshold tidal volume or a percentage drop in tidal volume. In at least one example, the at least one respiration sensor is a transthoracic impedance sensor and the percentage drop is 30-50%. In at least one example, the CSR-related condition is an apnea and wherein the IPG is configured to identify the apnea based at least in part on a determination of an absence of patient respiration above a threshold value for a threshold amount of time; and provide the first delivery of the electrical stimulation to the phrenic nerve in response to the determination of the apnea, wherein the first delivery of the electrical stimulation includes asynchronous stimulation delivered at a pre-programmed stimulation rate.

In at least one example, the at least one patient sensor comprises at least one accelerometer and wherein the CSR-related condition is a reduction in one or more of patient motion or pitch below a resting threshold. In at least one example, the at least one accelerometer is at least one of an implanted accelerometer or an accelerometer coupled to an exterior surface of the patient. In at least one example, the at least one processor is configured to increase an energy of the electrical stimulation from an initial energy level up to a maximum energy level over the pre-programmed time period or over one or more successive stimulation delivery time periods. In at least one example, the at least one processor is configured to enter the non-sleep time period therapy operational mode based at least in part on a pre-programmed start time-of-day and a pre-programmed end time-of-day.

In at least one example, the at least one processor is configured to monitor a signal from at least one patient motion sensor indicative of one or more of patient movement or patient position, determine one or more of motion or position parameters based on the signal, and enter the non-sleep time period therapy operational mode based at least in part on the one or more of motion or position parameters. In at least one example, the at least one patient motion sensor comprises at least one of an implanted accelerometer or an accelerometer coupled to an exterior surface of the patient. In at least one example, the at least one processor is further configured to enter a sleep apnea therapy operational mode during a pre-determined sleep time period for the patient identified at least in part based on the pre-programmed start time-of-day and the pre-programmed end time-of-day.

In at least one example, the at least one processor is further configured to control the stimulation circuitry to deliver electrical stimulation during the pre-determined sleep time period at a higher energy than during the pre-determined non-sleep time period. In at least one example, the IPG comprises a hermetically sealed casing, and wherein the at least one processor, the at least one memory, and the stimulation circuitry are disposed within the hermetically sealed casing. In at least one example, the IPG comprises at least one battery disposed within the hermetically sealed casing, and wherein the at least one battery comprises one or more of a rechargeable battery or a non-rechargeable battery. In at least one example, the at least one battery comprises the rechargeable battery and the IPG comprises magnetic inductive charging circuitry configured to provide a recharging current to the rechargeable battery.

In at least one example, the IPG comprises a header coupled to the hermetically sealed casing, the header comprising one or more receptacles configured to couple to the at least one implantable stimulation lead configured to provide electrical stimulation to the phrenic nerve. In at least one example, the target tissue comprises an upper-airway nerve and the header comprises one or more receptacles configured to couple to at least one implantable stimulation lead configured to provide electrical stimulation to the upper-airway nerve. In at least one example, the one or more receptacles are configured to couple to the at least one implantable stimulation lead comprising a cuff electrode or a transvenous electrode.

In at least one example, the one or more receptacles are configured to couple to the at least one implantable stimulation lead comprising the at least one respiration sensor. In at least one example, the header comprises at least one receptacle configured to couple to at least one implantable sensing lead comprising the at least one respiration sensor. In at least one example, the IPG is configured to provide one or more of bilateral phrenic nerve stimulation or unilateral phrenic nerve stimulation. In at least one example, the at least one respiration sensor comprises a transthoracic-impedance sensor. In at least one example, the at least one processor is further configured to control the stimulation circuitry based at least in part on signals from at least one of an electromyography sensor, a cardiac cycle sensor, an airflow sensor, an internal pressure sensor, a pulse oximeter, an effort belt, or a peripheral arterial tone sensor.

In at least one example, the at least one processor is further configured to select a stimulation mode to treat the CSR in the awake patient, wherein the selected stimulation mode may be a same mode as or a different mode from a sleep disordered breathing stimulation mode. In at least one example, the selected stimulation mode is one of an asynchronous stimulation mode, a synchronous stimulation mode, or an apnea-triggered stimulation mode. In at least one example, the selected stimulation mode may be a same mode as the sleep disordered breathing stimulation mode but with at least one stimulation parameter that is different between the selected stimulation mode and the sleep disordered breathing stimulation mode.

### BRIEF DESCRIPTION OF THE DRAWINGS

Various aspects of at least one embodiment are discussed below with reference to the accompanying figures, which may not be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or substantially similar component that is illustrated in various figures may be represented by a like numeral. For purposes of clarity, not every component may be labeled in every figure. In the figures:

FIG. 1A illustrates a graph depicting an example of breathing that exhibits Cheyne-Stokes respiration (CSR) patterns according to an example;

FIG. 1B illustrates a block diagram of a disordered breathing therapy system according to an example;

FIG. 1C illustrates a block diagram of a 24-hour period for a patient according to an example;

FIG. 2 illustrates a patient implanted with the implantable treatment system according to an example;

FIG. 3 illustrates an example of implantable components of the implantable treatment system;

FIG. 4 illustrates a perspective view of a disordered breathing therapy system using external sensors according to an example;

FIGS. 5A and 5B illustrate processes of treating disordered breathing in a patient according to an example;

FIG. 6A illustrates a process of providing waking CSR therapy to a patient according to an example;

FIG. 6B illustrates a process of delivering electrical stimulation to a patient according to an example;

FIG. 6C illustrates a process of providing waking CSR therapy to a patient according to another example;

FIG. 6D illustrates a table depicting different stimulation scenarios according to various examples;

FIG. 7A illustrates a swim-lane diagram depicting operation of the processor according to an example;

FIG. 7B illustrates a swim-lane diagram depicting operation of the processor according to another example;

FIG. 7C illustrates a swim-lane diagram depicting operation of the processor according to another example;

FIG. 7D illustrates a swim-lane diagram depicting operation of the processor according to another example;

FIG. 8 illustrates an example of an electrical stimulation trace depicting various electrical stimulation parameters; and

FIG. 9 illustrates a block diagram of a computing device according to an example.

### DETAILED DESCRIPTION

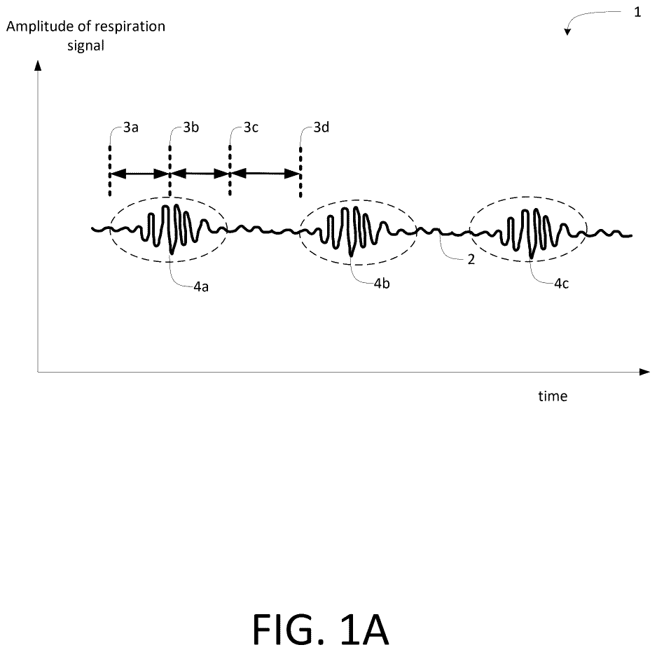

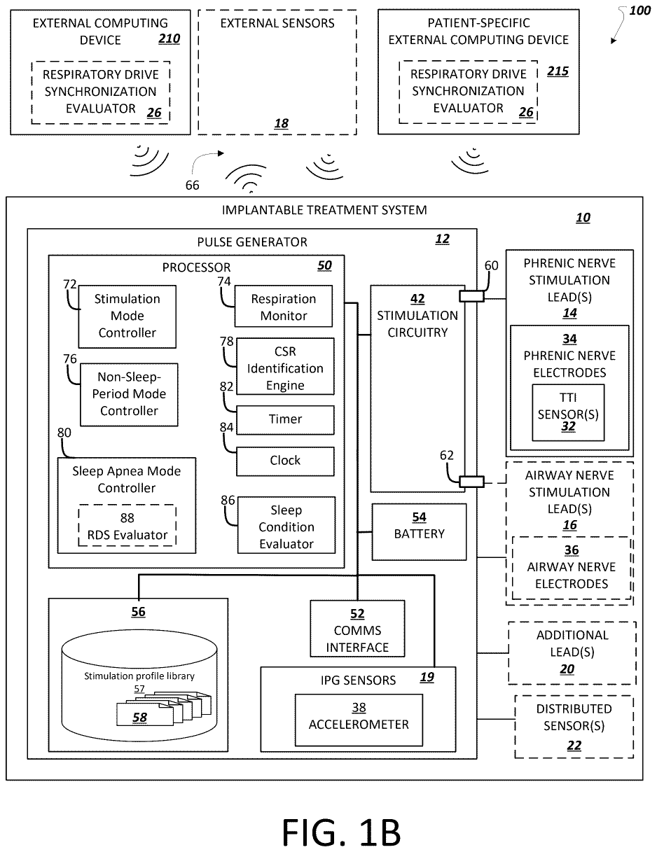

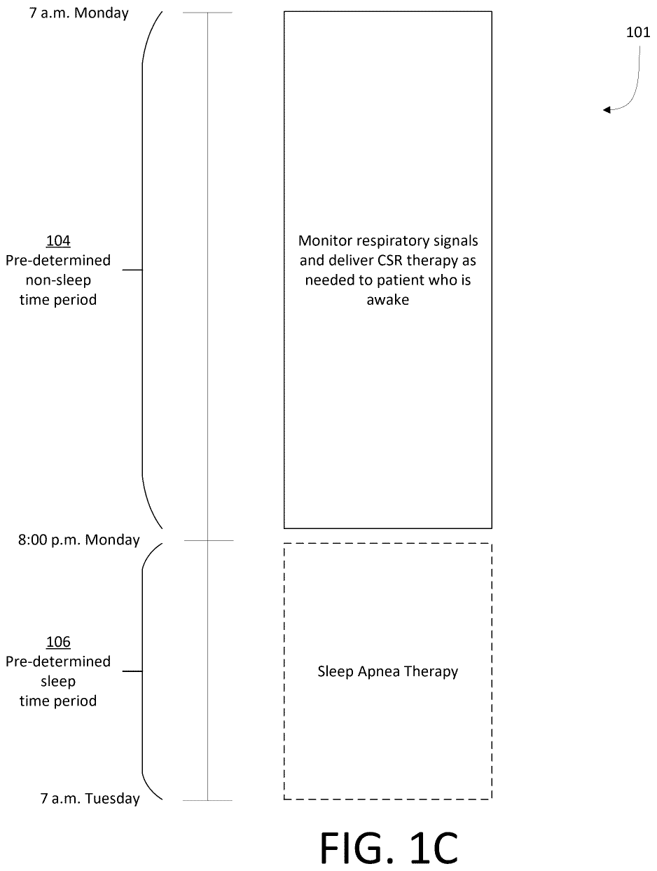

The term disordered breathing is used herein to describe a variety of observable respiration patterns that deviate from normal respiration. The term disordered breathing is used herein to describe a variety of observable respiration patterns that deviate from normal respiration including periodic breathing. One such observable respiration pattern is Cheyne-Stokes Respiration (CSR). For example, FIG. 1A illustrates a graph 1 depicting breathing that exhibits CSR patterns according to an example. The graph 1 includes an x-axis corresponding to time, a y-axis corresponding to an amplitude of respiration, and a trace 2 indicating a patient's breathing signal over time. As discussed above, CSR breathing may be characterized by repeated cycles of disordered breathing, each cycle including a period of rapid and/or deep breathing (for example, hyperpnea) followed by reductions in breathing (for example, apnea-hypopnea). The key feature of CSR is the reproducibility or similarity of the respiratory pattern across cycles of CSR.

To illustrate an example cycle of CSR breathing, the graph 1 includes a first time 3a, a second time 3b, a third time 3c, and a fourth time 3d. A cycle of CSR breathing extends from the first time 3a to the fourth time 3d. The cycle of CSR breathing is divided into a hyperpnea period of rapid and/or deep breathing, which extends from the first time 3a to the third time 3c, and a hypopnea (or apnea) period of reduced breathing from the third time 3c to the fourth time 3d.

During the hyperpnea period, an amplitude of the patient's breathing increases from the first time 3a to the second time 3b, then decreases from the second time 3b to the third time 3c. The period from the first time 3a to the second time 3b, during which the amplitude of the patient's breathing is increasing, may be referred to as a waxing period. The period from the second time 3b to the third time 3c, during which the amplitude of the patient's breathing is decreasing, may be referred to as a waning period. A first CSR envelope 4a, corresponding to an envelope of the trace 2 during the waxing and waning periods, outlines the amplitude of the signal and is roughly ovoid in shape. The pattern in the respiration signal from the first time 3a to the fourth time 3d may be referred to as a CSR pattern.

Subsequent CSR cycles also have a similarly shaped envelope during periods of hyperpnea, including a second CSR envelope 4b during a next CSR cycle, and a third CSR envelope 4c during a subsequent CSR cycle. Accordingly, in various examples, breathing that exhibits CSR patterns may include a period of hyperpnea, as indicated by the CSR envelopes 4a, 4b, 4c, followed by a period of apnea or hypopnea.

Examples of the disclosure provide stimulation therapy for CSR that presents while a patient is awake. CSR may occur in an awake person and/or in a sleeping person. CSR may be a patterned type of disordered breathing whether the patient is awake or asleep, though CSR that occurs while a patient is asleep may also be referred to as a patterned type of sleep apnea. The CSR pattern of breathing may occur intermittently between periods of normal breathing. Providing stimulation therapy to treat CSR in an awake patient presents challenges that do not affect stimulation therapy to treat CSR in a sleeping patient. Patients may engage in certain activities while awake that are not conducive to stimulation, such as talking, eating, drinking, singing, intentionally controlling one's breath during breath-control exercises, holding one's breath, exercising, and so forth. Stimulating the patient to affect the patient's breathing in these examples may lead to unintended adverse consequences for the patient.

Using eating as an example, the bodily mechanisms involved in eating may affect breathing in various ways. Structures in the oral cavity, pharynx, and larynx function in complex coordination to facilitate breathing, speaking, chewing and swallowing while a patient is eating. Food and air share the same spaces while chewing but must pass through different pathways when swallowing. Muscles of the larynx, glottis and epiglottis seal the airway to the lungs during swallowing, preventing aspiration of food and drink while also preventing passage of air into or out from the lungs. Accordingly, if the patient's breathing were stimulated while the patient is eating, the patient may inhale in response to the stimulation and may aspirate food or drink. This is only one example of the activities that a patient may be engaged in while awake for which stimulation may cause problems.

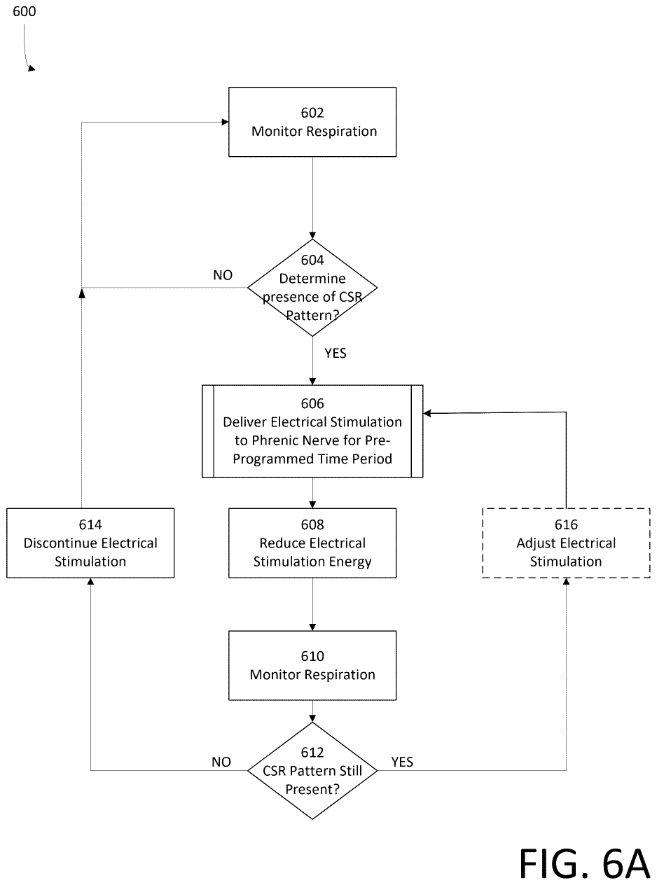

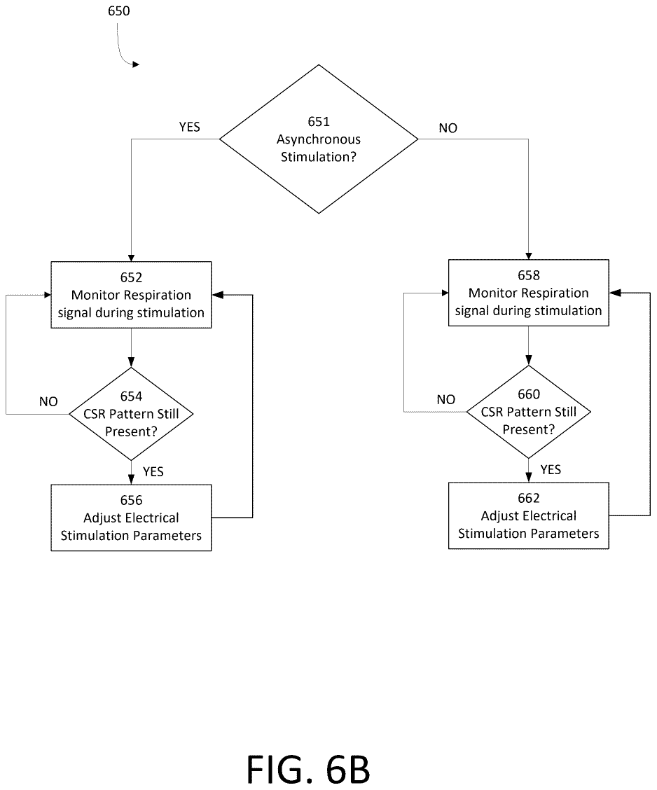

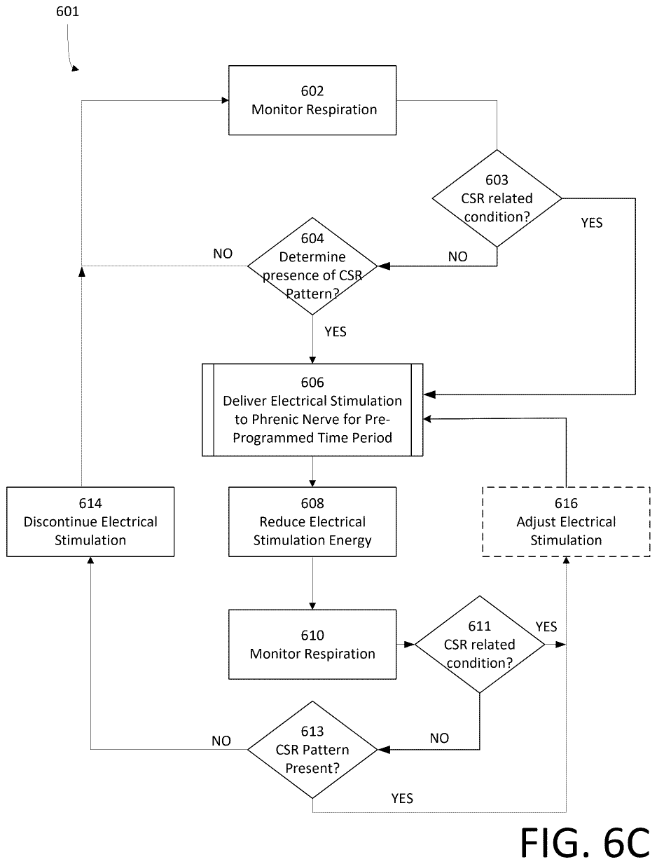

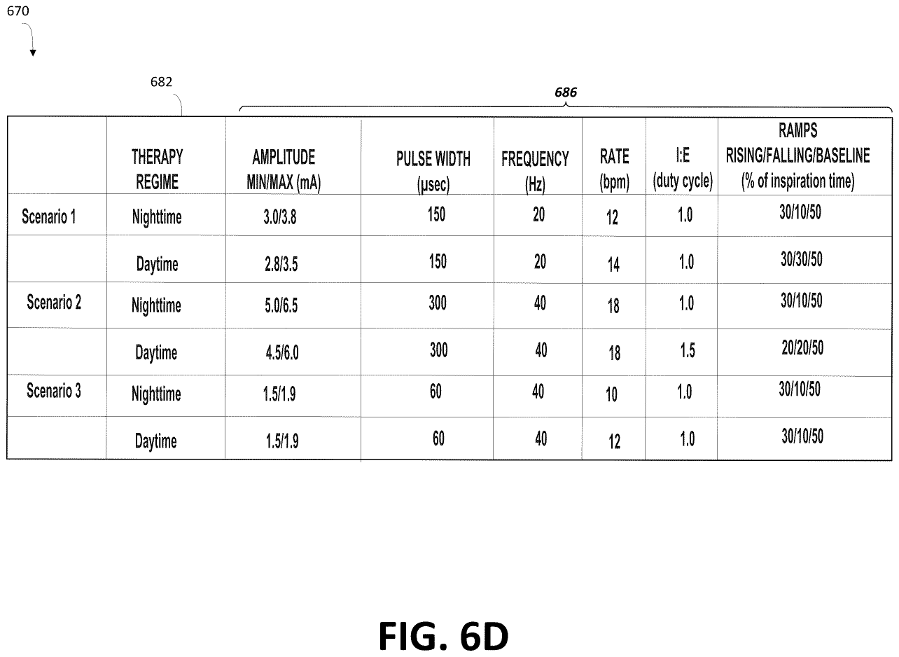

Additionally, at night, ongoing sleep apnea stimulation therapy may regulate breathing so that CSR does not occur. As discussed in more detail below following the description of FIG. 6B, in contrast to stimulation therapy delivered to a sleeping patient, in some examples, the system described herein may terminate or temporarily suspend monitoring for CSR and delivering CSR therapy responsive to one or more criteria being met that indicate a likelihood that the patient is engaged in certain activities for which stimulation is not desirable. During stimulation therapy to a sleeping patient and/or for a device that only delivers stimulation therapy when patient conditions indicate that the patient is asleep, the device and the stimulation therapy need not account for periodic breathing due to patient activities of the awake patient. These activities may include exercising, activity, talking, eating, etc.

In at least one example, a patient may be associated with a pre-determined sleep time period during which the patient is expected to be asleep and/or a pre-determined non-sleep time period during which the patient is expected to be awake. If the current time is outside of the pre-determined sleep time period and/or within the pre-determined non-sleep time period, stimulation therapy for wakeful CSR may be applied if the patient's breathing exhibits a CSR pattern. Example devices may determine that the patient's breathing exhibits a CSR pattern based on various techniques, such as a cross-correlation analysis between successive breathing cycles, an analysis of an amount of amplitude variation between breaths, a fast Fourier transform (FFT) analysis of a breath signal, and so forth. Accordingly, examples discussed herein may accurately identify, and provide stimulation therapy for, CSR that presents while a patient is awake. In various examples, stimulation may be suspended and/or altered based on a determination that the patient is engaged in activities for which stimulation may be disadvantageous.

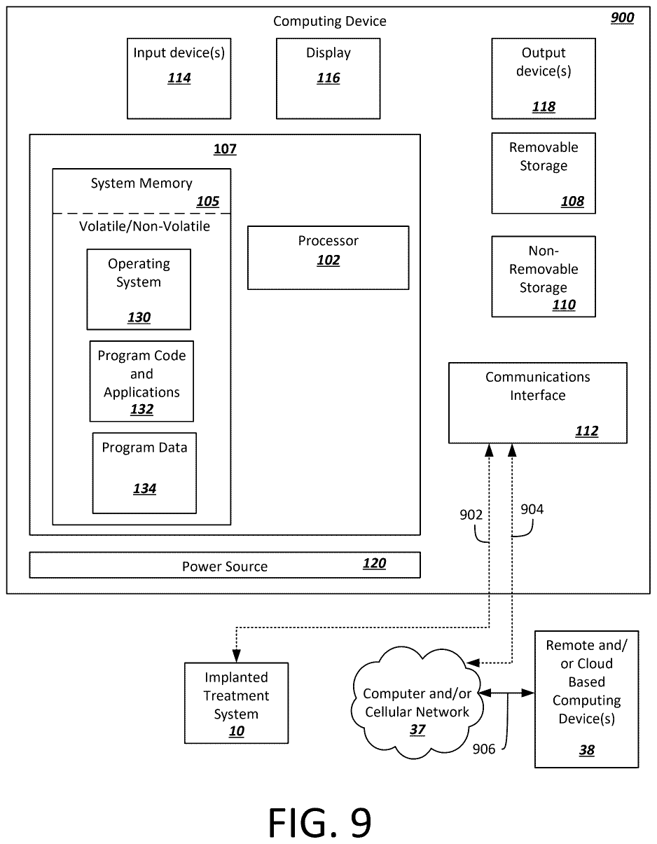

FIG. 1B illustrates a block diagram of a disordered breathing therapy system 100 according to an example. A quantity of each component in FIG. 1B is an example only and other quantities of each, or any, component could be used. The disordered breathing therapy system 100 may be implemented in connection with a patient to treat disordered breathing that occurs while the patient is awake, such as waking CSR (that is, CSR that presents while the patient is awake). Waking CSR may also be referred to as daytime CSR for purposes of convenience; however, daytime CSR may be understood to refer to waking CSR, and some patients may experience waking CSR during the nighttime. No limitation as to a time of day is thus implied by the term daytime CSR. In some examples, the system 100 may provide stimulation therapy to a patient who also experiences disordered breathing while the patient is asleep, such as CSA, OSA, and/or combination apneas, in addition to providing stimulation therapy to a patient who experiences CSR events while awake. FIG. 1B is described in part below and in part following the description of FIG. 9.

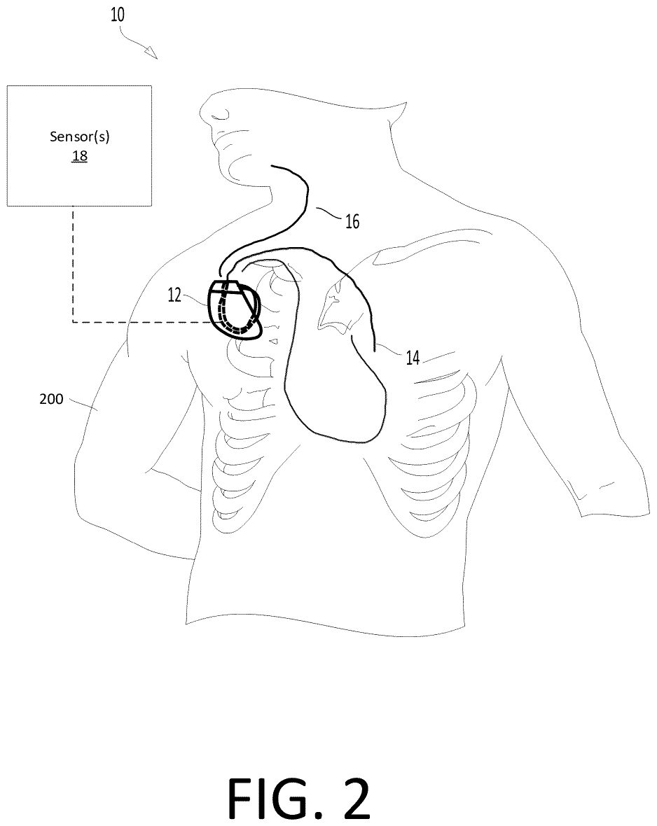

The disordered breathing therapy system 100 includes an implantable treatment system 10, an external computing device 210, one or more external sensors 18, and a patient-specific external computing device 215. The implantable treatment system 10 includes an implantable medical device 12 (for example, an implantable pulse generator 12, or IPG 12), one or more implantable stimulation leads (e.g., one or more phrenic nerve stimulation leads 14 and/or one or more airway nerve stimulation leads 16), and, optionally, one or more optional additional leads 20 and/or one or more optional distributed sensors 22. The implantable device 12 may be configured to be implanted in a patient. For example, FIG. 2 illustrates an example of a patient 200 implanted with the implantable treatment system 10 according to an example.

The IPG 12 may include IPG sensors 19, stimulation circuitry 42, at least one processor 50, a wireless communication interface 52, at least one power supply 54, and memory 56. The processor 50 may include a timer 82 and a clock 84, and is configured to include and/or execute a stimulation mode controller 72, a respiration monitor 74, a non-sleep-period mode controller 76, a CSR identification engine 78, a sleep apnea mode controller 80, and a sleep condition evaluator 86. The sleep apnea mode engine may optionally include and/or execute a respiratory drive synchronization (RDS) evaluator 88.

The timer 82 may time certain periods of time. For example, as discussed below, CSR stimulation therapy may be provided in one or more sets of stimulation each delivered over a pre-determined period of time which the timer 82 may time. The clock 84 may count time over a lifetime of the processor 50 and may correlate to a real time of day and date.

The stimulation mode controller 72, the respiration monitor 74, the non-sleep-period mode controller 76, the CSR identification engine 78, the sleep apnea mode controller 80 (including the RDS evaluator 88), the timer 82, the clock 84, and the sleep condition evaluator 86 may include hardware (for example, one or more controllers configured to execute computer-executable instructions and/or one or more hard-wired circuits), software and/or firmware (for example, instructions stored on non-transitory computer-readable media that, when executed by hardware such as the one or more controllers, causes the treatment system 10 to deliver therapy), or a combination thereof.

In some examples, the actions performed by the stimulation mode controller 72, the respiration monitor 74, the non-sleep-period mode controller 76, the CSR identification engine 78, the sleep apnea mode controller 80 (including the RDS evaluator 88), the timer 82, the clock 84, and the sleep condition evaluator 86 be implemented by hardware, software, firmware, microcode, hardware description languages, and so forth, or any combination thereof. When implemented in software, firmware, or microcode, the program code or code segments to perform the tasks may be stored in a non-transitory processor-readable medium such as a storage medium. One or more processors, including the processor 50, may perform the described tasks. In an implementation, the at least one processor 50 may include one or more integrated circuits and/or at least one application specific integrated circuit (ASIC).

The wireless communications interface 52 may include an electromagnetic telemetry transceiver along with at least one antenna for telemetry-based data communication and programming. Alternatively or additionally, the wireless communications interface 52 may include one or more of a transmitter, receiver, and/or transceiver configured for wireless communications, for example, according to a Bluetooth® communications protocol or other short-range communications protocol. In various implementations, the stimulation circuitry 42 and the at least one processor 50 may support dual-channel operations to support stimulation of a combination of at least one upper-airway nerve and at least one phrenic nerve.

The treatment system 10 is configured to be communicatively coupled to the computing system 210 via a wireless communication connection 66. Wireless communications may be exchanged pursuant to a short-range wireless communication protocol such as, for example, the Bluetooth® communication protocol, or inductive telemetry, or another telemetry method. The computing system 210 may program and/or configure the IPG 12.

The IPG 12 may receive programmable operating parameters via software and/or firmware downloaded to the IPG 12 via wired or wireless communications. In an implementation, the external computing device 210 may generate the programmable operating parameters, for example, based at least in part on user input to the external computing device 210. These programmable operating parameters may include one or more of stimulation energy and/or energy titration parameters. In some examples, the memory 56 may store one or more stimulation profiles 58 that specify programmable operating parameters and/or the electrical stimulation parameters thereof, as discussed in greater detail below. The IPG 12 may store the one or more stimulation profiles 58 in a stimulation profile library 57. Over the course of therapy provided to the patient, the stimulation mode controller 72 may select a desired stimulation profile from the stimulation profile library 57. The stimulation mode controller 72 may titrate stimulation provided to a patient, such as by selecting a different stimulation profile from the stimulation profile library 57 or by adjusting parameters of the stimulation in accordance with titration parameters dictated by a selected stimulation profile.

Various electrical circuitry components of the IPG 12, including one or more of the processor 50, the stimulation circuitry 42, the memory 56, and the battery 54, may be enclosed in a hermetically sealed casing to protect these components from the body environment. The IPG 12 may further include a header disposed on the casing. The header may be a structural component of the IPG 12 that provides one or more receptacles, or connector ports, configured to receive respective connector assemblies for leads connected to electrodes. Each of the leads 14, 16, 20 may couple with one or more connector ports.

In at least one example, the IPG 12 may include a phrenic-nerve connector port 60 configured to couple the stimulation circuitry 42 to the phrenic nerve stimulation leads 14 and an upper-airway-nerve connector port 62 configured to couple the stimulation circuitry 42 to the upper-airway-nerve stimulation leads 16. The header thus provides paths to electrically and mechanically couple the leads 14, 16, 20 to the enclosed components of the IPG 12 without comprising the hermetic seal. The connector ports may be configured and/or re-configured as sensor ports, stimulation ports, sensor-and-stimulation ports, and so forth according to programmed configuration settings provided to the IPG 12 through the firmware and/or software. The firmware and/or software may configure the connector ports to control and/or designate various electrodes as anodes, cathodes, and/or anode-cathode pairs. For example, the phrenic nerve stimulation leads 14, which may be or include at least one implantable sensing lead by virtue of including at least one respiration sensor, such as a transthoracic impedance (TTI) sensor 32, may be configured to couple to at least one receptacle of the header and provide sensor information to the IPG 12.

The power source 54 provides power to one or more components of the implantable treatment system 10. The power source 54 may include an energy-storage device, such as a battery. In some examples, the power source 54 may be rechargeable. For example, the power source 54 may be configured to be wirelessly recharged by a wireless charger outside of the patient's body. The IPG 12 may include magnetic inductive charging circuitry configured to provide a recharging current to the rechargeable battery 54. In other examples, the power source 54 may include a non-rechargeable battery, or may include a type of power source other than a battery.

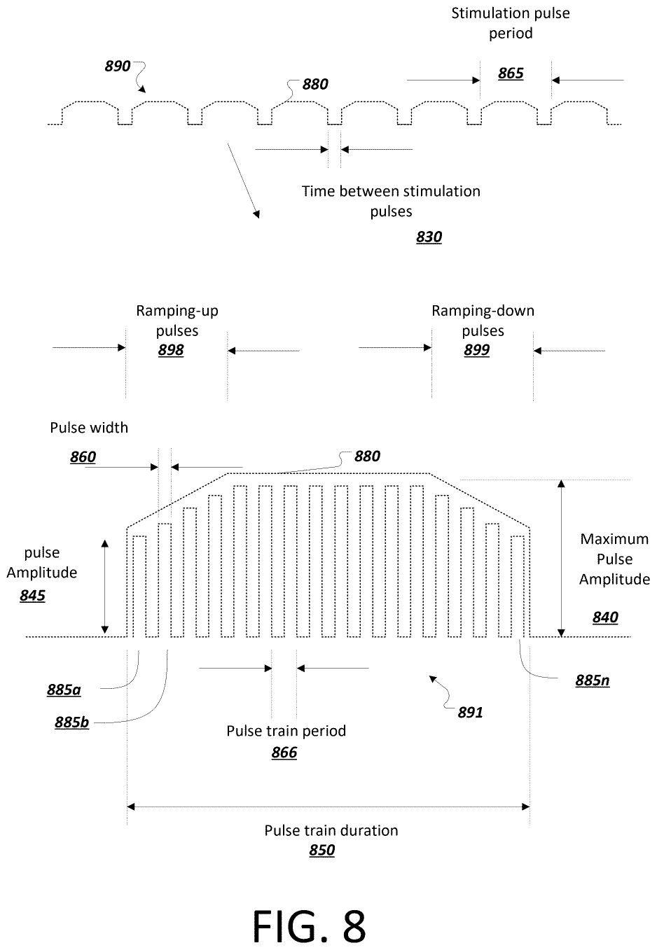

In various examples, the processor 50 is configured to process information and control operation of the treatment system 10, such as by controlling the delivery of electrical stimulation, for example, as one or more stimulation pulses, to a target nerve. Controlling the delivery of the electrical stimulation may include controlling operation of the stimulation circuitry 42. In some examples, the processor 50 may be configured to be able to select one or more stimulation parameters for controlling the operation of the stimulation circuitry 42, as discussed below with respect to FIG. 8. The processor 50 may include one or more processors to execute instructions stored on one or more non-transitory computer-readable media, such as the memory 56. Accordingly, when the treatment system 10 or IPG 12 is described as determining information or parameters, executing operations such as comparing values to thresholds, modifying parameters for electrical stimulation, sensing or obtaining parameters or information, and so forth, the processor 50 may be performing at least some of these operations.

The memory 56 may include one or more memory devices, one or more storage devices, or a combination thereof. For example, the memory 56 may include one or more non-transitory computer-readable media. The one or more non-transitory computer-readable media may store instructions that, when executed by the processor 50, cause the processor 50 to execute operations discussed herein, such as delivering waking CSR therapy. The memory 56 may also store other information, such as sensed data, user preferences, control commands from a physician, and so forth.

In operation, electrical stimulation pulse(s) generated by the IPG 12 travel along the leads 14 and/or 16. The leads 14 and/or 16 include electrodes to deliver the electrical stimulation to a target nerve. For example, the phrenic nerve stimulation leads 14 include phrenic nerve stimulation electrodes 34 and the airway nerve stimulation leads 16 include upper-airway nerve stimulation electrodes 36. Stimulation pulse(s) provided for a period of time to an electrode results in electrical stimulation of the nerve or other target tissue. The energy of the stimulation and the duration of the period of time, potentially amongst other factors, determine the stimulation power provided to the target tissue. The electrical stimulation activates the fibers (that is, generates an action potential) causing a functional response by the nerve. A higher energy stimulation may activate more nerve fibers resulting in a larger functional response. A lower energy stimulation may activate fewer nerve fibers resulting in a smaller functional response. In various examples, the IPG 12 may be configured to provide bilateral phrenic nerve stimulation and/or unilateral phrenic nerve stimulation.

For a particular functional response, such as the inducement of the contraction of a muscle (for example, the diaphragm), a minimum threshold energy may be required. The strength of the functional response (for example, the strength and/or extent of a muscular contraction needed to achieve a desired effect on respiration) determines the minimum threshold energy. In order to provide a range of functional responses (for example, a range of strengths and/or extents of muscular contractions to create a range of effects on respiration), a device, such as the IPG 12, may be configured to provide electrical stimulation to satisfy various energy thresholds. For example, there may be weak, medium, and strong thresholds to describe a range of functional responses.

As discussed in greater detail below, the disordered breathing therapy system 100 may include the RDS evaluator 88 and the RDS evaluator 26. As shown in FIG. 1B, the implantable treatment system 10 may include the RDS evaluator 88 and the external computing device 210 and/or the patient-specific external computing device 215 may include the RDS evaluator 26. For example, the sleep apnea mode controller 80 may include or execute the RDS evaluator 88.

In an implementation, the implantable treatment system 10 may include the RDS evaluator 26 in addition to or in lieu of the external computing device(s) 210, 215. The RDS evaluator 26 may provide initial programming of the RDS evaluator 88 during an initialization phase of use of the system 100 with the patient (for example, in physician-office visits designed to provide initial set-up of the implantable device 12). The RDS evaluator 88 may provide respiratory drive therapy as described herein and may modify respiratory drive treatment parameters based on RDS data to improve or otherwise modify synchronization. In some examples, the RDS evaluator 26 may periodically update or re-program the RDS evaluator 88 (for example, during follow-up visits in a physician's office).

The evaluators 88 and 26 may include hardware (for example, one or more controllers configured to execute computer-executable instructions), software and/or firmware (for example, instructions stored on non-transitory computer-readable media that, when executed by hardware such as the one or more controllers, causes the treatment system 10 to deliver therapy), or a combination thereof. In some examples, the actions performed by evaluator 88 and/or 26 may be implemented by hardware, software, firmware, microcode, hardware description languages, and so forth, or any combination thereof. When implemented in software, firmware, or microcode, the program code or code segments to perform the tasks may be stored in a non-transitory processor-readable medium such as a storage medium. One or more processors, including the processor 50, may perform the described tasks.

The processor 50 also includes the clock 84. The clock 84 may be configured to provide current time information, which may include a time of day, a day of the week, a month of the year, or another time indicator or clock information. In an implementation, the IPG 12 may use or evaluate the current time information to control delivery of electrical stimulation signals to the leads 14 and/or 16.

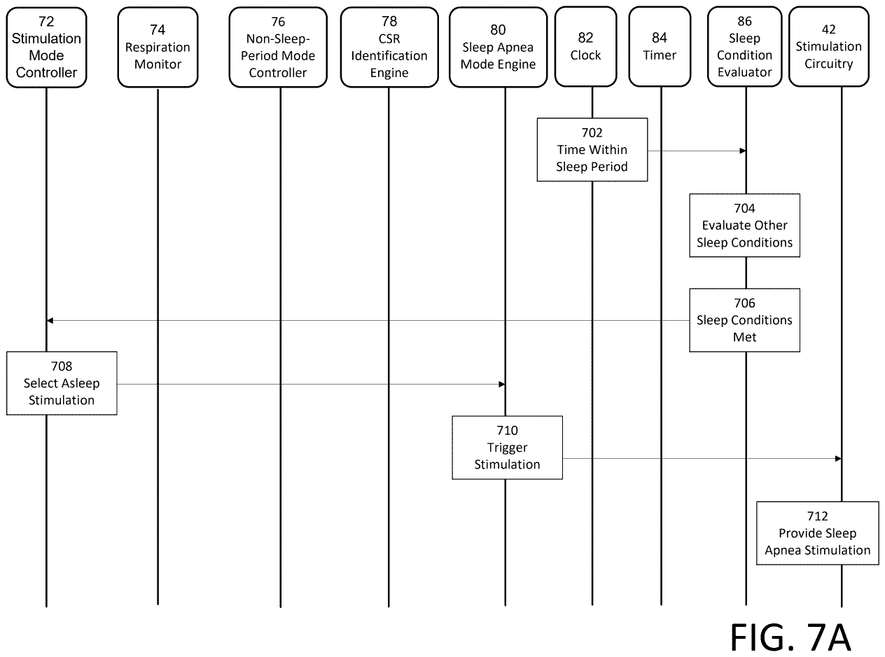

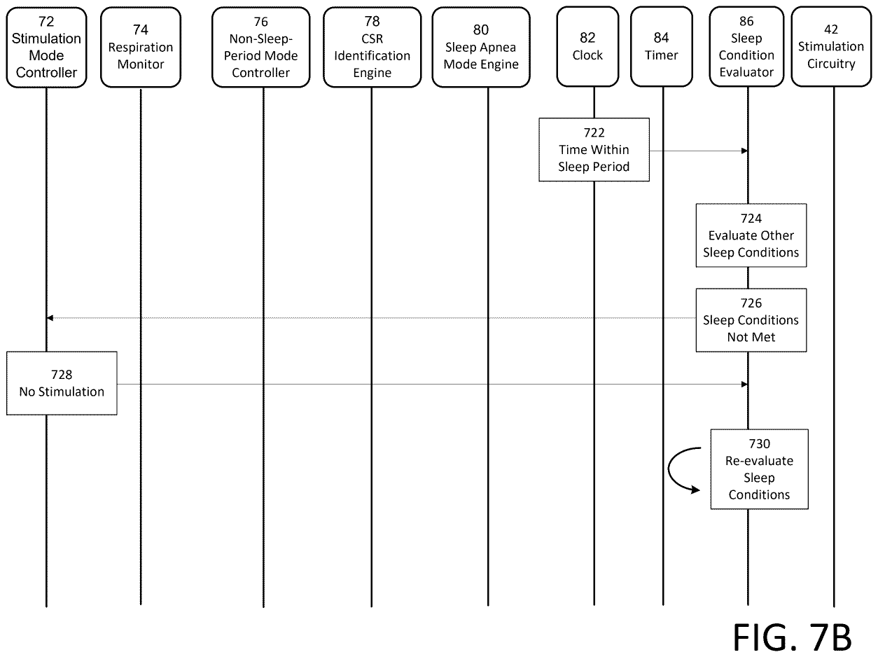

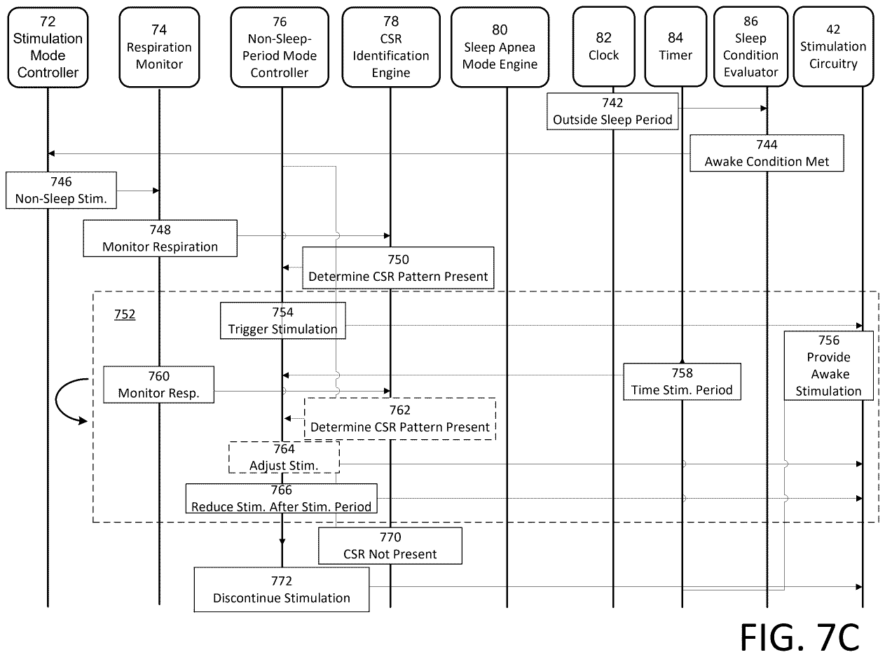

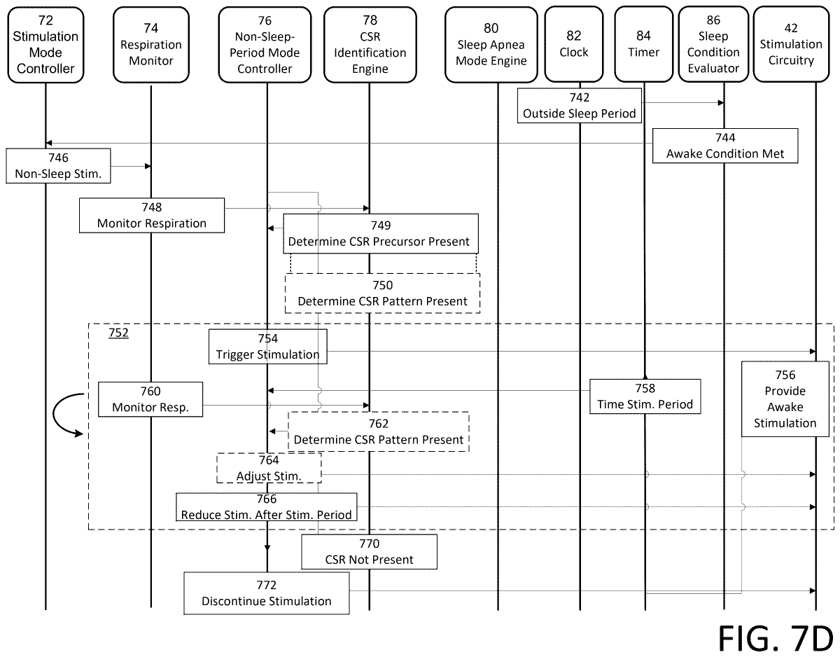

The processor 50 also includes the timer 82. The timer 82 may be configured to generate timing information, such as by counting down from an amount of time in response to one or more triggering conditions being met, or by counting up until an amount of time has elapsed in response to one or more triggering conditions being met. The amount of time may be a pre-determined amount of time. For example, as discussed below with respect to FIGS. 6A-7D, in some examples electrical stimulation may be provided for a pre-determined amount of time. The timer 82 may be used to determine when the pre-determined amount of time has elapsed.

The IPG 12 may receive sensed signals (e.g., one or more sensor signals) from one or more sensors, such as the external sensors 18, the IPG sensors 19, and/or the distributed sensors 22. Each of the sensors 18, 19, and/or 22 may include various types and/or numbers of sensors. The sensors 18, 19, and/or 22 may include one or more sensors such as one or more of motion sensors (for example, accelerometers), pressure sensors (for example, internal pressure sensors), acoustic sensors, transthoracic-impedance sensors, light sensors, airflow sensors, microphones, ultrasonic transducers, heart rate sensors, blood oxygenation sensors (for example, pulse oximeters), muscular electrical activity sensors (for example, electromyography sensors), peripheral arterial tone sensors, cardiac cycle sensors, or other types of sensors. The processor 50 may control the stimulation circuitry 42 based at least in part on signals from one or more of these sensors. In some examples, one or more of the leads 14 and/or 16 may include and/or act as sensors. For example, the phrenic nerve electrodes 34 may include one or more TTI sensors 32. Implanted and/or external accelerometers, such as the accelerometer 38, may be single axis accelerometers, or may be two- or three-axis accelerometers.

The external sensors 18 are external to the patient (that is, not implanted). The external sensors 18 may be wearable or otherwise coupled to the patient. The external sensors 18 may be communicatively coupled to one or more of the implantable device 12 and the external computing device 210, and may be electrically coupled to the external computing device 210. The external sensors 18 may provide sensed information to the IPG 12 via the wireless communication connection 66. For example, wireless communications may be exchanged pursuant to a short-range wireless communication protocol such as, for example, the Bluetooth® wireless communication protocol, or inductive telemetry, or another telemetry method.

In at least one example, any one or more of the sensors 18, 19, and/or 22 may include transthoracic-impedance sensors configured to measure an impedance across a patient's thorax. Furthermore, in some examples, the phrenic nerve electrodes 34 include the TTI sensor(s) 32. The transthoracic-impedance sensor data may be indicative of, for example, chest size, chest motion (that is, changes in chest size), cardiac parameters, and/or respiratory parameters that may be indicative of a degree of arousal and/or patient motion. For example, patient motion may include a patient activity like coughing or snoring that correlate with a change in degree of arousal. As another example, the transthoracic impedance data may be indicative of a change in lung volume due to changes in the respiratory pattern of a patient caused by a change in patient position. Thus, while the transthoracic impedance may not directly measure patient motion in the manner of an accelerometer, the transthoracic impedance may serve as a proxy or predictive measurement of patient motion.

Transthoracic-impedance data may be representative of, and/or may be used to determine, a patient's respiratory rate (for example, based on a frequency of the signal from the transthoracic-impedance sensor) and/or a patient's tidal volume (for example, based on an amplitude of the signal from the transthoracic-impedance sensor). The external sensors 18 may additionally or alternatively include other sensors, such as one or more of a heart-rate sensor, an optical sensor, a pressure sensor, an effort belt, an electrocardiogram sensor, an electroencephalogram sensor, and/or other types of sensors.

The IPG sensors 19 may include one or more sensors on or within the IPG 12. The one or more sensors may include at least one implantable motion sensor. For example, the at least one motion sensor may be at least one accelerometer 38. The at least one accelerometer 38 is an implantable accelerometer as opposed to the external sensors 18. For example, the at least one accelerometer 38 may be housed within the IPG 12. The at least one accelerometer 38 may be configured to sense a position and/or movement of a patient. The at least one implantable motion sensor may provide one or more sensor signals indicative of patient activity to the IPG 12. The IPG sensors 19 may be implantable within a patient, at least because the IPG 12 may be implantable within a patient. Because the at least one accelerometer 38 may be implantable within a patient, the processor 30 may use the position and/or movement information sensed by the at least one accelerometer 38 to determine, for example, a position of a sleeping patient to determine when a patient rolls from one position to another and/or a pitch of a patient to determine when a patient sits up because they are awake.

The distributed sensors 22 may include one or more sensors electrically coupled to the IPG 12, but not physically located within a housing of the IPG 12. For example, the distributed sensors 22 may include one or more pressure sensors implantable within a patient. Pressure sensors implanted within a patient may measure changes in pressure caused by a patient breathing as the patient's lungs expand and contract, or may measure changes in pressure caused by a patient's heart beating as the patient's heart expands and contracts, or may measure other changes in pressure. The distributed sensors 22 may include at least one accelerometer in addition to, or in lieu of, other sensors, such as the pressure sensors. In an example, an accelerometer on a lead proximate to the hypoglossal nerve, or located elsewhere proximate to the airway, may detect motion indicative of airway patency. For example, at least one accelerometer may be implemented on one or more of the optional additional leads 20. The distributed sensors 22 may be electrically coupled to the IPG 12 to provide sensed information to the IPG 12.

The external computing device 210 may include, for example, a computing device operated by a physician. A physician may use the external computing device 210 to control and/or program the implantable treatment system 10. For example, the physician may use the external computing device 210 to control and/or program the RDS evaluator 88 to modify parameters of therapy delivered by the IPG 12. Those parameters may include parameters discussed in regard to FIG. 8, for example, one or more of a minimum stimulation energy, a maximum stimulation energy, a number of stimulation pulses, a frequency of stimulation pulses, a width of the stimulation pulses, a stimulation pulse amplitude, a maximum stimulation pulse amplitude, a minimum stimulation pulse amplitude, a stimulation current, a stimulation voltage, a stimulation polarity (for example, monophasic or biphasic), stimulation energy ramps, and so forth. As discussed in regard to FIG. 8, the stimulation pulses may be provided as pulse trains and therefore the parameters may include pulse train parameters.

The patient-specific computing device 215 may include, for example, a computing device operated by a patient. For example, the patient-specific computing device 215 may include a smartphone, tablet, laptop, wearable computing device (e.g., a watch [such as a smartwatch], an adhesive device, a headset, glasses, etc.), or other computing device operated by the patient. The patient may use the patient-specific computing device 215 to control and/or program the implantable treatment system 10. For example, the patient may use the patient-specific computing device 215 to control and/or program the RDS evaluator 88 to modify parameters of therapy delivered by the IPG 12, such as the parameters discussed above and with respect to FIG. 8. A patient may additionally or alternatively use the patient-specific computing device 215 to manually pause or activate therapy. In some examples, a physician operating the external computing device 210 may be able to modify a different number of parameters (for example, a greater number of parameters), and/or may be able to modify parameters within a different range of values (for example, a broader range of values), than the patient operating the patient-specific computing device 215.

As discussed above, the system 10 may provide stimulation therapy to patients experiencing CSR events while awake in addition to, or in lieu of, patients experiencing sleep apnea events. In some examples, the system 10 may associate a patient with a pre-determined sleeping period during which the patient is usually asleep, and/or a pre-determined non-sleeping period during which the patient is usually awake.

FIG. 1C illustrates a block diagram of a 24-hour period 101 for a patient according to an example. In the illustrated example, the patient's day may be divided between a pre-determined non-sleeping period 104 and a pre-determined sleeping period 106. The pre-determined non-sleeping period 104 may be a pre-determined period during which the patient is expected to be awake, and the pre-determined sleeping period 106 may be a pre-determined period during which the patient is expected to be asleep. The patient may select the time ranges of the periods 104, 106, which may be different for each day, depending on when the patient is typically awake and asleep. The pre-determined non-sleeping period 104 may start based on a pre-programmed start time-of-day, and may end based on a pre-programmed end time-of-day. The pre-determined sleeping period 106 may start based on a pre-programmed end-of-day, and may end based on a pre-programmed start-of-next-day.

Although the terms day and night may be used, the pre-determined non-sleeping period 104 may occur primarily during the nighttime if, for example, the patient is typically awake at night and asleep during the day. Accordingly, references to the pre-determined non-sleeping period 104 as occurring during the day are provided for convenience rather than limitation, as the pre-determined non-sleeping period 104 may occur primarily during the night in some patients.

The periods 104, 106 may collectively span a 24-hour period. For example, the pre-determined non-sleeping period 104 may span a 13-hour period from 7 AM to 8 PM on a Monday, and the pre-determined sleeping period 106 may span an 11-hour period from 8 PM on Monday to 7 AM on Tuesday. The patient may select the periods 104, 106 based on an expectation that they are typically awake from 7 AM to 8 PM on Monday, and are typically asleep between 8 PM on Monday and 7 AM on Tuesday. In other examples, different periods of time may be implemented. For example, the patient's pre-determined sleeping period may be later on the weekends than on weekdays. In some examples, a patient may be associated with multiple sleeping periods and/or multiple non-sleeping periods within a 24-hour period. For example, a patient may have a primary sleeping period during which the patient sleeps for a long period of time, and at least one secondary sleeping period during which the patient sleeps for a shorter period of time. The shorter period(s) of time may include, for example, times during which a patient typically naps. The patient may have non-sleeping periods between these sleeping periods. For example, a first non-sleeping period may extend from 7 AM to 4 PM on Monday, a first sleeping period may extend from 4 PM to 5 PM on Monday (for example, corresponding to a nap), a second non-sleeping period may extend from 5 PM to 8 PM on Monday, and a second sleeping period may extend from 8 PM on Monday to 7 AM on Tuesday.

During the pre-determined non-sleeping period 104, the system 100 may monitor respiration signals and deliver CSR stimulation therapy as needed to a patient who is awake. As discussed above, in monitoring the respiration signals the system 100 may be configured to distinguish between respiration signals that exhibit CSR (in response to which CSR stimulation therapy may be provided), and respiration signals that are indicative of daily waking activities such as drinking, talking, exercising, and so forth (in response to which CSR stimulation may not be provided).

During the pre-determined sleeping period 106, the system 100 may optionally deliver sleep apnea therapy. In other examples, the system 100 may not provide any therapy during the pre-determined sleeping period 106. For example, for a patient who only experiences waking disordered-breathing events and does not experience sleep apnea events, the system 100 may not be programmed to provide any therapy during the pre-determined sleeping period 106. Conversely, for a patient who experiences both waking disordered-breathing events and also experiences sleep apnea events, the system 100 may be programed to provide sleep apnea therapy during the pre-determined sleeping period 106 (for example, in response to a determination that the patient is experiencing a sleep apnea event).

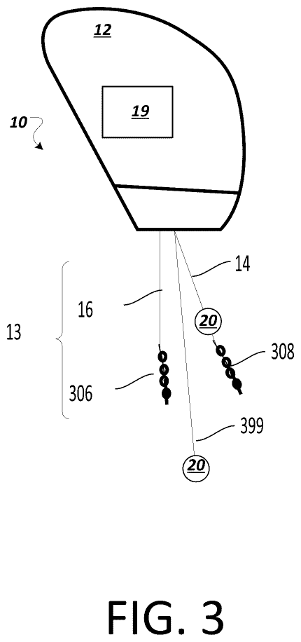

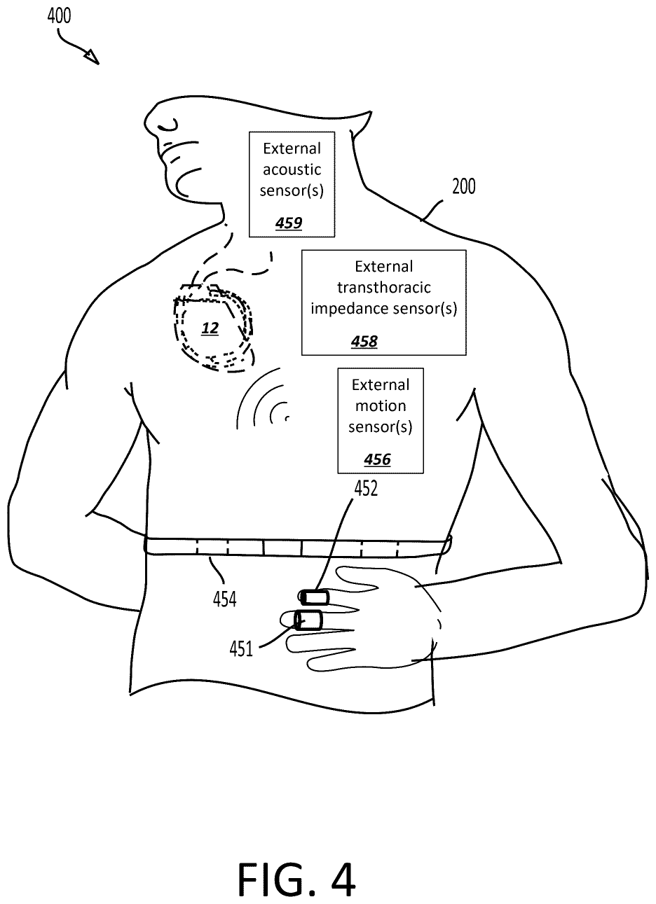

Accordingly, the system 100 may monitor respiration signals and determine, based on the respiration signals, whether to provide stimulation therapy to a patient. Respiration signals may be provided by one or more sensors. FIGS. 3 and 4 illustrate examples of aspects of the system 100.

FIG. 3 illustrates an example of implantable components of the implantable treatment system 10. A quantity of each component in FIG. 3 is an example only and other quantities of each, or any, component could be used.

As illustrated in FIG. 3, the implantable components 10 include leads 13, which include the phenic nerve stimulation leads 14 and the upper-airway stimulation leads 16, and the IPG 12. The phrenic nerve electrodes 34 include a plurality of phrenic nerve electrodes 306, and the airway nerve electrodes 36 include a plurality of upper-airway nerve electrodes 308. The electrodes 306 and/or 308 may be transvenous electrodes and/or nerve cuff electrodes. The electrodes 306 and/or 308 may serve as distributed sensors 22 and/or the stimulation leads may include distributed sensors 22. In some examples, the implantable components may include leads 399 without stimulation electrodes but which include the distributed sensors 22. For example, the optional additional leads 20 may include the leads 399.

The one or more phrenic nerve leads 14 may be configured for implantation in a blood vessel proximate to the phrenic nerve or may include a nerve cuff configured for implantation around the phrenic nerve. The one or more upper-airway stimulation leads 16 may be configured for implantation in a blood vessel proximate to the hypoglossal nerve or may include a nerve cuff configured for implantation around the hypoglossal nerve. For example, the leads 14, 16 may be sized and/or shaped in a configuration that allows the leads 14, 16 to be implanted in, and remain stable in, a respective target blood vessel without damaging the respective target blood vessel. Alternatively or additionally, the leads 16 may include leads configured for implantation in a blood vessel proximate the ansa cervicalis or other upper-airway nerve or may include a nerve cuff configured for implantation around the ansa cervicalis or other upper-airway nerve. Where a nerve has a left and right occurrence, the leads 13 may be configured to service one or both of the occurrences (for example, the left phrenic nerve, the right phrenic nerve, or both). In some examples, the leads 13 may be considered unilateral leads when servicing one occurrence of the nerve, and may be considered bilateral leads when servicing both occurrences of the nerve.

The IPG 12 may control the electrical energy delivered to the leads 13 to treat decreased respiratory drive (that is, to cause the diaphragm to contract due to stimulation of the phrenic nerve) and/or to improve or restore airway patency (that is, to cause movement of muscles in the upper-airway due to stimulation of an upper-airway nerve such as, for example the hypoglossal nerve). For example, the IPG 12 may deliver electrical stimulation to one or more target nerves via the phrenic nerve stimulation lead(s) 14 and/or the airway nerve stimulation lead(s) 16. Such target nerves may include, for example, one or more of a phrenic nerve, a hypoglossal nerve, the ansa cervicalis, other nerves that activate a diaphragm and/or modify a position of airway tissue which may include the tongue, and so forth.

In various examples, the IPG 12 may deliver electrical stimulation signals to one or more of the leads 13. The leads 13 may each include electrodes to deliver electrical stimulation to a target nerve based on the electrical stimulation signals. For example, the processor 50 may cause the stimulation circuitry 42 to deliver the electrical stimulation to the leads 13. The processor 50 causing the stimulation circuitry 42 to deliver stimulation may include controlling one or more switching elements, such as one or more metal-oxide-semiconductor field-effect transistors (MOSFETs), bipolar junction transistors (BJTs), relays, diodes, other switching elements, a combination thereof, and so forth, within the stimulation circuitry 42 to modulate power (for example, derived from the power supply 54) and produce an electrical stimulation with desired electrical stimulation parameters.

FIG. 4 illustrates a perspective view of a disordered breathing therapy system 400 using external sensors according to an example. A quantity of each component in FIG. 4 is an example only and other quantities of each, or any, component could be used. The disordered breathing therapy system 400 may be an example of the disordered breathing therapy system 100 in an implementation including one or more external sensors 18. For example, the one or more external sensors 18 may include one or more of a pulsatility sensor 452, an effort belt 454, external motion sensor(s) 456, external transthoracic impedance sensors 458, and/or external acoustic sensor(s) 459, and so forth.

The external motion sensor(s) 456 may include at least one external accelerometer. In various implementations, the external motion sensor(s) 456 may be affixed to the body of the patient at one or more locations (for example, chest, torso, neck, head, legs, arms, and so forth). These external motion sensor(s) 456 may provide one or more sensor signals indicative of patient activity to the IPG 12. Further, the external motion sensor(s) 456 may have a gravity reference and the IPG 12 may filter the signals from the external motion sensor(s) 456 to remove direct current (DC) offset. The at least one external accelerometer may measure vibrations of a patient's tissues. The effort belt 454 may also include one or more external accelerometers.

In various implementations, the effort belt 454 is implemented to detect breathing of the patient 200, and the at least one pulsatility sensor 452 is implemented to measure pulsatility parameters, such as cardiac pulse rate, heart rate, pulse amplitude fluctuations, a peripheral arterial tone, and/or SpO2, and so forth. The external sensors 18 may include a pulse oximeter 451.

The implantable device 12 may also be coupled to one or more of the effort belt 454, the at least one transthoracic impedance sensor 458, and/or the at least one pulsatility sensor 452. The pulsatility sensor 452, the effort belt 454, the motion sensor(s) 456, the at least one transthoracic impedance sensor 458, and/or the external acoustic sensor(s) 459 may be examples of the external sensors 18. The external sensors 18 may be communicatively coupled to the implantable device 12 in some examples, either directly (for example, via the wireless communication connection 66 with the implantable device 12) or indirectly (for example, by communicating with the computing system 210 which, in turn, communicates with the implantable device 12).

The effort belt 454 may be coupled around the chest of the patient 200. As the patient 200 breathes, the effort belt 454 may deform. The effort belt 454 may include one or more pressure and/or stretch sensors to detect changes in pressure applied to the effort belt 454 and/or stretching of the effort belt 454 as a result of the patient 200 breathing. Accordingly, the effort belt 454 may sense parameters indicative of the breathing of the patient 200 which may be used to determine information such as a breathing rate of the patient 200.

The at least one transthoracic impedance sensor 458 may be coupled to, and external to, a chest of the patient 200. The at least one transthoracic impedance sensor 458 may sense an impedance across the chest of the patient 200. The measured impedance may vary based on the size and/or dimensions of the chest cavity of the patient 200 which may, in turn, vary as the patient 200 breathes. Accordingly, the at least one transthoracic impedance sensor 458 may sense parameters indicative of the breathing of the patient 200 which may be used to determine information such as a breathing rate of the patient 200.

Transthoracic impedance data may be indicative of, for example, chest size, chest motion (that is, changes in chest size), cardiac parameters, and/or respiratory parameters that may be indicative of a degree of arousal and/or patient motion. For example, patient motion may include a patient activity like coughing or snoring that correlates with a change in degree of arousal. As another example, the transthoracic impedance data may be indicative of a change in lung volume due to changes in the respiratory pattern of a patient caused by a change in patient position. Thus, while the transthoracic impedance may not directly measure patient motion in the manner of an accelerometer, the transthoracic impedance may serve as a proxy or predictive measurement of patient motion.

The at least one pulsatility sensor 452 may be coupled to a finger of the patient 200. The at least one pulsatility sensor 452 may track pulsatility parameters of the patient 200, such as pulsatile volume changes in peripheral arterial beds as the heart of the patient 200 beats and/or as the patient 200 breathes. Accordingly, the at least one pulsatility sensor 452 may sense parameters indicative of the breathing of the patient 200 which may be used to determine information such as a breathing rate, heart rate, blood-oxygen concentration, and so forth, of the patient 200.

With regard to the effort belt 454, in an implementation, the IPG 12 may use motion data from the belt 454 to assess entrainment, presence of respiratory effort, respiratory rate, and so forth. Based on this assessment the IPG 12 may initiate, terminate, reduce, or increase stimulation to the phrenic nerve stimulation leads 14 and/or the airway nerve stimulation leads 16 to titrate the treatment provided to the patient 200. Similarly, the IPG 12 may assess data from the transthoracic impedance sensor(s) 458, the external motion sensor(s) 456, and/or the pulsatility sensor(s) 452 in order to initiate, terminate, reduce, or increase stimulation to the one or more of the leads 13 to titrate the treatment provided to the patient.

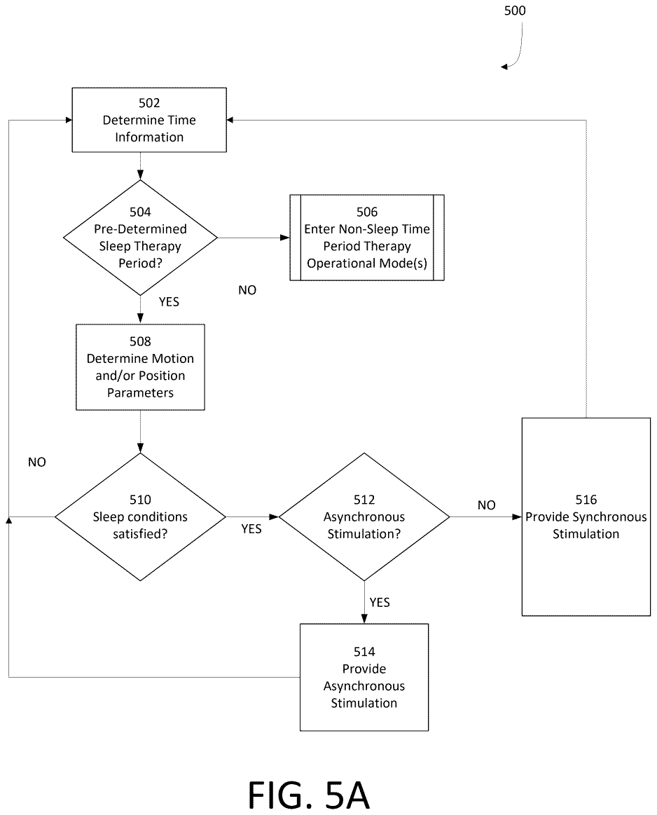

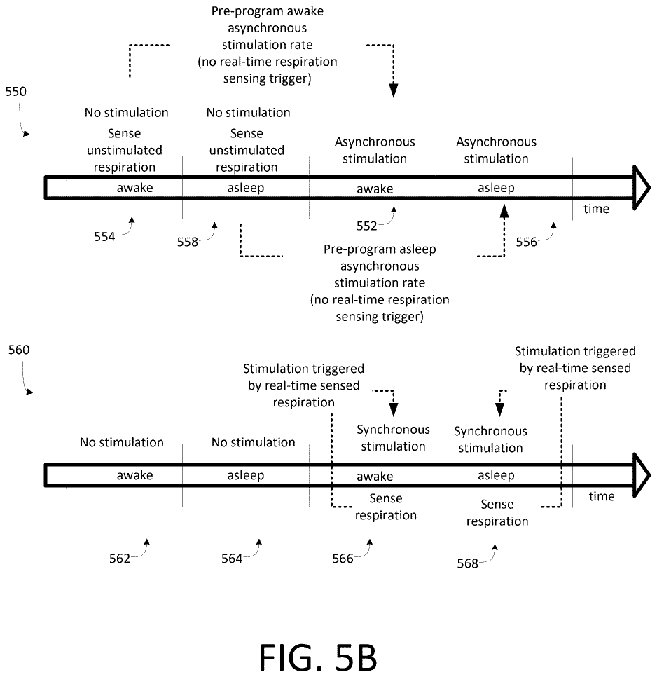

FIG. 5A illustrates a process 500 of treating disordered breathing in a patient according to an example. The process 500 may be executed at least in part by the processor 50. For purposes of example, the process 500 may provide an example in which the system 100 provides stimulation therapy for patients experiencing both waking CSR and sleep apnea. As discussed above, the system 100 may in other examples provide stimulation therapy for patients experiencing only waking CSR without also experiencing sleep apneas.

At act 502, the processor 50 determines time information. Time information may include a time of day and a day of the week. The clock 84 may provide the time information to the processor 50. For example, the processor 50 may poll the clock 84 for time information.

At act 504, the processor 50 determines based on the time information whether the current time is within a pre-determined sleeping period. As discussed above with respect to FIG. 1C, a patient may be associated with at least one pre-determined sleeping period (for example, the pre-determined sleeping period 106) and/or at least one pre-determined non-sleeping period (for example, the pre-determined non-sleeping period 104). Information indicative of the pre-determined sleeping period and/or the pre-determined non-sleeping period may be stored in the memory 56. As discussed above, the sleeping and non-sleeping periods may be selected by a patient and/or a practitioner (and stored in the memory 56) based on the patient's typical expected sleeping time. Act 504 may therefore include the processor 50 determining whether the time information indicates that the current time is within a pre-determined sleeping period or a pre-determined non-sleeping period.

If the processor 50 determines that the current time is not within a pre-determined sleeping period (and, in some examples, is thus within a pre-determined non-sleeping period) (504 NO), then the process 500 continues to act 506.

At act 506, the processor 50 enters a non-sleep time therapy operational mode. During the non-sleep time therapy operational mode, the processor 50 may provide CSR therapy to the awake patient. Examples of the non-sleep time therapy operational mode are discussed below with respect to FIGS. 6A-7D. As discussed in greater detail below, the CSR therapy may include various types of synchronous or asynchronous therapy. Furthermore, in some examples, the system 10 may transition between therapy modes and/or may deliver therapy with different therapy parameters.

Otherwise, if the processor 50 determines that the current time is within a pre-determined sleeping period (and, in some examples, is thus not within a pre-determined non-sleeping period) (504 YES), then the process 500 continues to act 508.

At act 508, the processor 50 determines one or more motion and/or position parameters of the patient. Motion parameters may include parameters indicative of the patient moving, such as accelerometer data indicative of the patient walking around. Position parameters may include parameters indicative of a position of the patient, such as whether the patient is lying down on their back, lying down on their side, lying down on their stomach, or standing up, and/or may include a pitch of the position, such as whether the patient is sitting up. The processor 50 may receive the motion and/or position parameters from one or more of the sensors 18, 19, 22. For example, the processor 50 may receive motion and/or position parameters from the accelerometer 38.

At act 510, the processor 50 determines whether the one or more motion and/or position parameters satisfy one or more sleep indication conditions. Sleep indication conditions may include conditions which, if satisfied, indicate that the patient is likely asleep. The one or more motion and/or position parameters may serve as a proxy for the actual sleep state of the patient. Patient motion and/or position may be indicative of sleep particularly when combined with a time of day that corresponds to a regular sleeping time of the patient. For example, if the motion parameters indicate that the patient is walking around (and the time of day is within a non-sleeping period), the patient is likely awake. Conversely, if the motion parameters indicate that the patient is not moving, the position parameters indicate that the patient is lying down, and the time of day is within a sleeping period, the patient is more likely to be asleep.

Thus, the sleep indication conditions may include pre-programmed thresholds or ranges for patient position and/or patient movement. Optionally, the sleep indication conditions may include a patient position (for example, a patient position in one of four positional quadrants including a left-side position, a right-side position, a prone position, and a supine position) and/or changes in physiological parameters, such as a reduction in heart rate.

If the processor 50 determines that the sleep indication conditions are not satisfied (510 NO), then the process 500 returns to act 502. Acts 502-510 may be repeated until the processor 50 determines that the current time is no longer within a pre-determined sleeping period (504 NO), or the processor 50 determines that the one or more motion and/or position parameters satisfy the sleep indication conditions (510 YES). If the processor 50 determines that the one or more motion and/or position parameters satisfy the sleep indication conditions (510 YES), then the process 500 continues to act 512.

At act 512, the processor 50 determines whether to enter an asynchronous stimulation mode or a synchronous stimulation mode. Asynchronous stimulation and synchronous stimulation differ in how a stimulation is triggered. As discussed further in regard to FIG. 5B, in synchronous stimulation, respiration sensed in real-time triggers the provision of stimulation by the IPG 12 for each breath cycle. Thus, the stimulation rate is responsive in real-time to the sensed respiration and is synchronized with that sensed respiration. Asynchronous stimulation is provided at a rate that is pre-determined and/or pre-programmed prior to the provision of stimulation and is thus triggered by a pre-determined and/or pre-programmed setting of the IPG 12. The stimulation in an asynchronous mode is not triggered by respiration sensed at each breath cycle.

In some examples, the asynchronous stimulation mode may include providing stimulation in the presence or absence of an apnea. In a different asynchronous stimulation mode, referred to as an apnea-triggered asynchronous stimulation mode, the stimulation rate may similarly be pre-determined and/or pre-programmed; however, the stimulation may be provided only in response to detecting an apnea as a CSR-related condition as discussed below in regard to FIG. 6C. In various examples, a patient may be experiencing an apnea when the patient's respiration remains below a threshold value for at least a threshold period of time. For example, a patient may be experiencing an apnea if the amplitude of the patient's respiration does not exceed a threshold value within, say, 3-10 seconds. In the apnea-triggered asynchronous stimulation mode, the system 10 may not provide stimulation unless and until the system 10 detects such a patient apnea. Asynchronous stimulation may therefore refer to a type of stimulation that may be provided in an asynchronous stimulation mode or an apnea-triggered asynchronous stimulation mode.

Accordingly, while the asynchronous stimulation mode, the apnea-triggered asynchronous stimulation mode, and the synchronous stimulation mode may be entered in response to the same or similar criteria (for example, the one or more sleep indication conditions being satisfied at act 510—YES) in some examples, the stimulation modes may differ as discussed with respect to FIG. 5B. Stimulation may be provided on either a synchronous or asynchronous basis in various different examples and, in some examples, the system 10 may switch between synchronous and asynchronous stimulation.

FIG. 5B illustrates an asynchronous stimulation diagram 550 and a synchronous stimulation diagram 560. Each of the diagrams 550, 560 depicts actions performed by the system 10 as a function of time, which elapses from left to right in FIG. 5B. As shown in FIG. 5B, the implantable therapy system 10 may provide the electrical stimulation according to one of various stimulation therapy modes including an asynchronous therapy mode (which may include an asynchronous stimulation mode in which stimulation is provided whether or not an apnea is present, and/or an apnea-triggered asynchronous stimulation mode in which stimulation is provided in response to an apnea being detected) or a synchronous therapy mode. The non-sleep period mode controller 76 may select the stimulation therapy mode for non-sleep time stimulation and the sleep apnea mode controller 80 may select the stimulation therapy mode for the sleep time stimulation. The system 10 may not implement these modes simultaneously but may operate in one mode at a time with the capability of changing between modes based on programming and/or other settings. Selection of the stimulation therapy mode may occur prior to and/or after the system 10 is implanted in the patient. The system 10 may be configured to change between modes automatically based on programming and/or other settings and/or may change between modes in response to instructions from the external computing device 210 and/or the patient-specific external computing device 215.

Each of the asynchronous mode and the synchronous mode may be used for awake stimulation or for sleeping stimulation, such as a sleep disordered breathing stimulation mode. However, while the mode may be the same for an awake period as for a sleep period, stimulation parameters (examples of which are provided below with respect to the stimulation parameters 686 of FIG. 6D) may not be the same for an awake period and a sleep period. For example, the pre-determined or pre-programmed asleep asynchronous stimulation rate may be the same or different than the pre-determined or pre-programmed awake asynchronous stimulation rate. Similarly, the rate at which sensed respiration triggers synchronous stimulation in an asleep patient may be rate the same or different than for the awake patient.

In the asynchronous stimulation mode corresponding to the asynchronous stimulation diagram 550, the system 10 provides the phrenic nerve stimulation at a predetermined and/or pre-programmed stimulation rate based on intrinsic respiration rate sensed in the absence of stimulation and at a time prior to the application of stimulation. The system may then determine the pre-programmed stimulation rate to be equal to the intrinsic rate or one, two, three, four, or another number of breaths-per-minute higher or lower than the intrinsic rate. The delivery of stimulation for each breath cycle will then be provided independently of (that is, not triggered by) respiration sensed in real-time during the provision of therapy.

The time prior to the application of stimulation (that is, at times 554 or 558) may be immediately prior, such as within a particular sleeping period or non-sleeping period, or may be a time removed from the particular sleep or non-sleeping period (for example during a surgical recovery period before the patient is released for home use, or at a doctor office visit between uses, and so forth). For example, the system 10 may determine a stimulation rate for therapy provided to an awake patient 552 based on a sensed unstimulated respiration rate (for example, an awake intrinsic rate) prior to applying therapy while the patient is awake 554, and may determine a stimulation rate for therapy provided to an asleep patient 556 based on a sensed unstimulated respiration rate (for example, an asleep intrinsic rate) prior to applying therapy while the patient is asleep 558. In an example, the time prior to the application of stimulation (that is, at the times 554 and 558) may be subsequent to surgical implantation of the system 10 and during a healing and observation period before the device is activated for at-home use by the patient. For the awake patient, the time prior to the application of stimulation may be a time when the patient is awake but prior to a detection of CSR and/or a CSR-related condition, for example a CSR precursor (as discussed in regard to FIGS. 6A-6D). The time prior to the application of stimulation may include a monitoring period (corresponding to times 554 and 558) in which the system 10 monitors and detects intrinsic respiration for the purpose of determining an asynchronous stimulation rate. Thus, in the asynchronous mode the implantable treatment system 10 is configured to provide electrical stimulation in response to a preprogrammed or predetermined stimulation rate trigger that excludes respiration sensed concurrently with the applied stimulation therapy.

In the synchronous stimulation mode corresponding to the synchronous stimulation diagram 560, respiration sensed in real-time during stimulation triggers the provision of the phrenic nerve stimulation by the system 10. That is, while a period 562 prior to applying therapy during which a patient is awake and a period 564 prior to applying therapy during which a patient is asleep may exist, the patient's intrinsic respiration during these periods 562, 564 may not directly control a stimulation rate as there is no stimulation available to trigger during these time periods.

The synchronous stimulation mode provides breathing support and the sensed respiration while stimulation therapy is being provided triggers the stimulation. For example, the system 10 may provide stimulation to an awake patient at a stimulation rate that is based on the patient's sensed respiration while the patient is awake at that time 566, and may provide stimulation to an asleep patient at a stimulation rate that is based on the patient's sensed respiration while the patient is asleep at that time 568.

In this manner, the stimulation is synchronous with the patient's respiration cycles during stimulation. Thus, the synchronous mode is a respiration triggered stimulation mode. The synchronous mode may further include providing apnea-triggered stimulation at an asynchronous rate in response to or based on a lack of a sensed respiration in real-time. For example, the system 10 may determine that there is a lack of a sensed respiration if no respiration signal above a certain threshold is received within a certain threshold period of time (for example, five seconds, eight second, 10 seconds, 15 seconds, and so forth). Such an apnea-triggered stimulation at an asynchronous rate may be an automatic feature of the synchronous therapy mode as a patient safety feature so that a patient experiencing a lack of respiration does not remain untreated by the system 10. The system 10 may deliver stimulation in response to detecting the lack of the sensed respiration rather than waiting indefinitely for a sensed respiration trigger. The system 10 may provide the apnea-triggered stimulation at a pre-determined and/or pre-programmed rate, that is, an asynchronous rate. This rate may be based on previously sensed intrinsic patient respiration or may be based on a population average or other clinically derived effective stimulation rate.

FIG. 5B shows asynchronous stimulation provided for both the awake and asleep periods on one timeline and synchronous stimulation provided for both the awake and asleep periods on another timeline, but this representation is for simplicity and brevity only. In various examples, as discussed in regard to FIG. 6D, consecutive awake and asleep periods may also use different stimulation therapy modes.

Returning to act 512 of FIG. 5A, the system 10 may determine whether to enter an asynchronous stimulation or synchronous stimulation mode which, as discussed above, may be based on programming and/or other settings and/or may be based on instructions from the external computing device 210 and/or the patient-specific external computing device 215. For a given patient, the system 10 may switch between asynchronous and synchronous stimulation at various times. For example, the system 10 may provide asynchronous or synchronous stimulation while the patient is awake, and may provide asynchronous or synchronous stimulation while the patient is asleep, but the stimulation mode provided while the patient is awake may or may not be the same as the stimulation mode provided while the patient is asleep.

If the system 10 selects an asynchronous stimulation mode (512 YES), then the process 500 continues to act 514. Otherwise, if the system 10 selects a synchronous stimulation mode (512 NO), the process 500 continues to act 516.

At act 514, the system 10 enters an asynchronous stimulation mode in which asynchronous stimulation may be provided to a patient. As discussed below, asynchronous stimulation may be provided based on different conditions being met, and may include different stimulation parameters in different examples. The process 500 then returns to act 502.

At act 516, the system 10 enters a synchronous stimulation mode in which synchronous stimulation may be provided to a patient, optionally with apnea-triggered stimulation functionality included as a safety feature. As discussed below, synchronous stimulation may be provided based on different conditions being met and may include different stimulation parameters in different examples. The process 500 then returns to act 502.

Although asynchronous or synchronous therapy may be provided to either a waking or sleeping patient, acts 514 and 516 may include entering at least one sleep-apnea-therapy operational mode due to the conditions evaluated at acts 502, 504, and 508. That is, the asynchronous and synchronous therapy modes of acts 514 and 516 (corresponding to the patient being asleep) may be sleep-apnea-therapy modes. Sleep-apnea-therapy operational modes may include a mode of operation in which the system 100 provides CSA therapy, a mode of operation in which the system 100 provides OSA therapy, a mode of operation in which the system 100 provides combination therapy, such as mixed apnea therapy, and so forth. The disordered breathing treated during sleep may include CSR. As discussed above, different patients may experience different types of sleep apneas, and the system 100 may be configured to treat the type(s) of sleep apnea that the patient is experiencing. Acts 514 and/or 516 may include the sleep apnea mode controller 80 controlling the stimulation circuitry 42 to deliver, via the leads 14 and/or 16, electrical stimulation to a patient to treat one or more forms of sleep apnea.

In some examples, the energy of the electrical stimulation provided at acts 514 and 516 may differ from the energy of the electrical stimulation provided at act 506. For example, acts 514 and 516 may include the IPG 12 delivering electrical stimulation with a higher energy than at act 506. Because the patient is awake at act 506, the patient may be more likely to notice electrical stimulation than while asleep. Accordingly, the IPG 12 may provide lower-energy electrical stimulation at act 506 as compared to acts 514 and 516. Examples of different stimulation parameters are provided below with respect to FIG. 6D.

As discussed above, in some examples the system 100 may be configured to provide CSR therapy only for a waking patient, and may not provide any sleep-disordered breathing therapy. For example, a patient may be treated for waking CSR but may not experience and/or be treated for sleep related breathing disorders. In examples in which the system 100 only provides waking CSR therapy, acts 508-516 may be omitted. If the processor 50 determines at act 504 that the current time is within a pre-determined sleep time period (504 YES), the modified version of the process 500 may return to act 502 rather than continuing to act 508.

Thus, in examples in which the system 100 only provides CSR therapy for patients who are awake, a corresponding version of the process 500 may include the processor 50 determining whether a current time is within a pre-determined sleeping period and taking no additional action if the current time is within the pre-determined sleeping period, but entering a non-sleeping period therapy mode of operation if the current time is not within the pre-determined sleeping period.

Accordingly, the processor 50 may execute the process 500 to determine, based on time information, whether the current time is within a pre-determined non-sleeping period. The processor 50, and thus the IPG 12 and the system 10, is therefore configured to be able to select a mode of operation to enter based at least in part on whether the current time is within a pre-determined non-sleeping period or is within a pre-determined sleeping period. In some examples, the processor 50 may be configured to be able to select a mode of operation based on additional or different parameters.

For example, in some examples the processor 50 may analyze patient position and/or motion parameters prior to entering the non-sleep time period therapy operational mode. That is, while in some examples the processor 50 may determine motion and/or position parameters responsive to determining that the current time is within a pre-determined sleep time period (504 YES), in other examples, the processor 50 may determine motion and/or position parameters prior to determining whether the current time is within the pre-determined sleep time period, or may determine motion and/or position parameters responsive to determining that the current time is not within the pre-determined sleep time period (504 NO). For example, the processor 50 may enter the non-sleep time period therapy mode based at least in part on determining that the patient position is not one of a list of sleeping positions.

If the processor 50 determines that the current time is within a pre-determined non-sleeping period, the processor 50 may monitor the patient's respiration to determine whether to provide waking CSR stimulation therapy as discussed below with respect to FIG. 6A.

FIG. 6A illustrates a process 600 of providing waking CSR therapy according to an example. The processor 50 may execute the process 600. In some examples, the processor 50 may execute one or more acts of the process 600 by utilizing one or more of the stimulation mode controller 72, the respiration monitor 74, the non-sleep-period mode controller 76, the CSR identification engine 78, or the sleep condition evaluator 86, as discussed in greater detail below with respect to FIGS. 7A-7D.

At act 602, the processor 50 monitors patient respiration by monitoring one or more respiration parameters. As discussed in greater detail below, the processor 50 may execute the respiration monitor 74 to monitor patient respiration. Monitoring the one or more respiration parameters may include monitoring a patient-respiration signal from at least one respiration sensor. For example, the trace 2 discussed above with respect to FIG. 1A provides an example of a patient-respiration signal.

The at least one respiration sensor may include at least one sensor that senses respiration parameters. Respiration parameters may include parameters indicative of a patient's respiration. For example, respiration parameters may include transthoracic-impedance data or information derived therefrom, such as a patient's breathing rate and/or tidal volume. In some examples, the processor 50 may receive transthoracic-impedance data from at least one respiration sensor, such as the TTI sensor 32, and may derive a patient's breathing rate and/or tidal volume from the transthoracic-impedance data. In various examples, however, the one or more respiration parameters may only include transthoracic-impedance data, and the processor 50 may not determine a patient's breathing rate and/or tidal volume from the transthoracic-impedance data. Accordingly, in some examples the processor 50 may directly analyze the transthoracic-impedance data without converting the data to, for example, a breathing rate and/or tidal volume. In various implementations, the respiration parameters may include airflow data, accelerometer data, pulse oximetry data, or parameters derived therefrom. One or more of the sensors 18 19, 22 may provide sensor signals indicative of the respiration parameters to the processor 50 and/or the respiration monitor 74.

At act 604, the processor 50 determines, based on the one or more respiration parameters, whether the patient respiration is indicative of a CSR pattern. As discussed in greater detail below, the processor 50 may execute the CSR identification engine 78 to determine whether the patient respiration is indicative of a CSR pattern. Determining whether the patient respiration is indicative of a CSR pattern may include determining whether a CSR pattern is present in the monitored respiration signal. In some examples, determining whether the patient respiration is indicative of a CSR pattern may include determining whether the CSR pattern is present in the monitored respiration signal for at least a threshold duration of time. In at least one example, the threshold period of time may include one or more complete cycles of a CSR pattern.

The processor 50 may therefore detect the CSR pattern by identifying multiple similar periodic breathing cycles. Whereas irregular breathing including caused by activities such as exercising, talking, eating, and so forth may be transient, a CSR pattern may exhibit as multiple cycles of breathing with similar properties (for example, similar waxing and waning properties). For example, the CSR envelopes 4a-4c discussed above with respect to FIG. 1A may represent at least portions of a CSR pattern and may each be similar to one another. In another example, the threshold duration of time may include either a certain number of complete cycles of the CSR patterns (for example, two or more CSR cycles) and/or an amount of time elapsing, such as between 30-120 seconds. The processor 50 may identify CSR at least in part based on implementation of a detection algorithm to determine the presence of the CSR pattern based on the monitored respiration signal.

In one example, the detection algorithm executed at act 604 may include a cross-correlation analysis. The processor 50 may execute a cross-correlation analysis to determine a correlation index between signal morphology elements (for example, CSR envelopes, like the CSR envelopes 4a-4c) of the respiration signal. If the morphology elements, such as the CSR envelopes, are very similar to one another (that is, the elements exhibit a high degree of cross-correlation and thus yield a high correlation index), the processor 50 may determine that CSR is present.

Based on the correlation index, the processor 50 may identify the signal morphology elements as CSR cycles, in which case act 604 may include a determination that the patient respiration is indicative of a CSR pattern (604 YES), or may identify the signal morphology elements as not being CSR cycles, in which case act 604 may include a determination that the patient respiration is not indicative of a CSR pattern (604 NO).

In some examples, the cross-correlation analysis may include the processor 50 monitoring the patient respiration signal(s) for a period of time. For example, the period of time may be at least 30 seconds, or at least one minute, or at least three minutes, or at least four minutes, or no more than five minutes, or at least five breath cycles, or some other amount of time. The processor 50 may generate a respiration trace, similar to the trace 2, from the monitored patient respiration signal. In some example, the processor 50 may normalize the magnitude of the trace, such as by scaling the trace such that a highest magnitude of the trace is set equal to 1, and the lowest magnitude of the trace is set equal to 0. In other examples, the processor 50 may not normalize the trace.

The processor 50 may identify a series of morphology elements within the trace. The morphology elements may include sections of the trace with similar morphologies to one another. For example, the morphology elements may include signal oscillations, such as those defining the CSR envelopes 4a-4c in FIG. 1A. In some examples, therefore, the processor 50 may identify a series of CSR envelopes in the trace. The processor 50 may then cross-correlate each morphology element with a respective subsequent morphology element. For example, in the context of FIG. 1A, the processor 50 may cross-correlate the first CSR envelope 4a with the second CSR envelope 4b, and may cross-correlate the second CSR envelope 4b with the third CSR envelope 4c.

Cross-correlating may include determining a cross-correlation index for each pair of morphology elements quantifying a degree of correlation between the successive morphology elements. The processor 50 may then evaluate the similarity between morphology elements based on the cross-correlation index. The processor 50 may compare the similarity to a similarity threshold to determine whether CSR is present. If the similarity exceeds the similarity index, the processor 50 may identify CSR.

In another example, the detection algorithm executed at act 604 may include an amplitude-variation analysis in addition to, or in lieu of, the cross-correlation analysis. As discussed above, a CSR cycle may be characterized at least in part by a waxing period (for example, from the first time 3a to the second time 3b) and a waning period (for example, from the second time 3b to the third time 3c). Accordingly, a large difference in amplitude values (for example, between a low-amplitude respiration signal at the first time 3a, when the waxing period begins, and a high-amplitude respiration signal at the second time 3b, when the waxing period ends) may be indicative of a CSR pattern.

The processor 50 may monitor the patient respiration signal and generate a trace indicative of the patient respiration as discussed above with respect to the cross-correlation analysis. The processor 50 may determine a variation in amplitude between a lowest-amplitude respiration signal and a highest-amplitude respiration signal. In some examples, the processor 50 may identify a CSR pattern if the variation in amplitude exceeds a threshold amount. The threshold amount may include, for example, relative values between the lowest-and highest-amplitude respiration signals, such as 50%, 65%, 80%, 90%, and so forth, or absolute values between the lowest-and highest-amplitude respiration signals, such as a threshold transthoracic-impedance value.

In some examples, the processor 50 may identify the CSR pattern if the respiration signal not only exhibits the variation in amplitude in excess of the threshold amount, but also if certain apneas are present in the respiration signal. In a first example, the processor 50 may identify the CSR pattern if the respiration signal satisfies the amplitude-variation criteria and one or more apneas are detected. For example, the processor 50 may identify an apnea if the respiration signal remains below a threshold value for at least a threshold amount of time, such as eight seconds, 10 seconds, 12 seconds, or some other amount of time.

In a second example, the processor 50 may identify the CSR pattern if the respiration signal satisfies the amplitude-variation criteria and one or more apneas are not only detected, but also occur at regular intervals. For example, the processor 50 may determine that the apneas occur at regular intervals if, during an evaluated period of time, the periods between successive apneas remain within a threshold range of time.

In a third example, the processor 50 may identify the CSR pattern if the respiration signal satisfies the amplitude-variation criteria and one or more apneas are not only detected, but also each occur following a respective waxing-and-waning period. For example, the processor 50 may determine that the apneas follow a respective waxing-and-waning period if, during an evaluated period of time, the processor 50 detects an apnea within a threshold amount of time of a respective waxing-and-waning period. In some examples, the processor 50 may identify the CSR pattern if the processor 50 determines that the respiration signal satisfies the amplitude-variation criteria and is indicative of at least a threshold number of apneas, such as two, three, four, or some other number of apneas, occur in a row following respective waxing-and-waning periods.

In another example, the detection algorithm executed at act 604 may include a frequency-domain analysis in addition to, or in lieu of, the cross-correlation analysis and/or the amplitude-variation analysis. As discussed above, a CSR cycle may be characterized at least in part by a waxing period (for example, from the first time 3a to the second time 3b) and a waning period (for example, from the second time 3b to the third time 3c). The processor 50 may monitor the patient respiration signal and generate a trace indicative of the patient respiration as discussed above with respect to the cross-correlation analysis. The processor 50 may execute an FFT algorithm to convert the respiration signal to the frequency domain. The processor 50 may perform a spectral analysis of the respiration signal in the frequency domain to identify one or more patterns of frequency that correspond to the waxing-and-waning pattern associated with CSR events.

Based on detecting the frequency signature(s) of the waxing-and-waning patterns, the processor 50 may perform a spectral analysis in the frequency domain to confirm that the waxing-and-waning patterns are regular, which may include determining whether a time between each successive waxing-and-waning period is within a threshold amount of time. Using FIG. 1A as an example, the processor 50 may execute an FFT algorithm to convert the trace 2 to the frequency domain, perform a spectral analysis to identify the waxing-and-waning pattern of the CSR envelopes 4a-4c, and perform a spectral analysis to determine whether a time between each successive one of the CSR envelopes 4a-4c is within a threshold amount of time.

Accordingly, act 604 may include the processor 50 executing one or more detection algorithms to determine, based on the respiration signal obtained at act 602, whether a CSR pattern is observable in the respiration signal. The one or more detection algorithms may include one or more of a cross-correlation analysis, an amplitude-variation analysis, a frequency-domain analysis, a combination thereof, and/or other types of analysis. The processor 50 may be configured to be able to perform one or more of the analyses, and may select a type of analysis to provide based, for example, on pre-programmed settings and/or based which analysis is most effective in detecting the presence of CSR.

In some examples, act 604 may include the processor 50 analyzing patient activity. As discussed in greater detail below, it may be advantageous to pause or discontinue electrical stimulation during periods of high patient activity. Stimulating the patient during periods of high activity may cause the patient to inhale or exhale at disadvantageous times, because the patient's exercise may lead to deep, irregular breathing. In some examples, the processor 50 may receive a signal indicative of patient activity from an accelerometer, such as the accelerometer 38 (which may be an implanted accelerometer) and/or an accelerometer of the external sensors 18 (which may include an external accelerometer coupled to an exterior surface of the patient, such as the patient's skin). In another example, the processor 50 may receive a signal indicative of patient activity from a heart-rate sensor, such as a heart-rate sensor of the distributed sensors 22 (which may include an implanted heart-rate sensor) and/or a heart-rate sensor of the external sensors 18 (which may include an external heart-rate sensor). The determination of the presence of the CSR pattern may therefore be based at least in part on a patient activity level, which may be based on patient movement, patient heart rate, and/or other parameters indicative of patient activity. For example, the processor 50 may suspend determining that the CSR pattern is present while the patient activity level exceeds a threshold patient activity level.

If the processor 50 does not determine the presence of a CSR pattern (604 NO), then the process 600 returns to act 602. Acts 602 and 604 may then be repeated until the processor 50 determines that a CSR pattern is present (604 YES), in which case the process 600 continues to act 606. In some examples, the processor 50 may store a CSR event marker indicative of the detected CSR event in the memory 56 responsive to detecting the CSR pattern (604 YES). The processor 50 may transmit the CSR event marker to one or both of the external computing devices 210, 215 via the wireless communications interface 52. In some examples, the processor 50 may output an alarm indicative of the CSR pattern determination to the patient-specific external computing device 215 responsive to determining the presence of the CSR pattern (604 YES).

In an implementation, the process 600 may not include the act 604 and may merely deliver therapy to an awake patient at one or more particular times during the patient's waking hours. The times may be pre-determined and/or may be patient initiated via an external controller for the implantable system 10. The duration of the therapy may be pre-determined and/or patient controlled. In a patient known to experience CSR during waking hours, such a system may proactively provide CSR therapy without needing to specifically detect the presence of the CSR pattern.

At act 606, the processor 50 controls the stimulation circuitry 42 to deliver a first delivery of electrical stimulation to a target tissue of the patient, such as at least one phrenic nerve, for a pre-programmed period of time. For example, the processor 50 may control one or more switching elements of the stimulation circuitry 42 to modulate power (for example, derived from the power supply 54) and produce an electrical stimulation with desired electrical stimulation parameters. The processor 50 (for example, the non-sleep-period mode controller 76) may control the stimulation circuitry 42 to deliver the electrical stimulation to the phrenic nerve electrodes 34 via the phrenic nerve stimulation leads 14 to stimulate the phrenic nerve of the patient. In some examples, as discussed below with respect to FIG. 6B, act 606 may include monitoring the respiration signal during the pre-programmed period of stimulation and, if the CSR pattern is still present, adjusting the electrical-stimulation parameters. As further discussed with respect to FIG. 6B, act 606 may include providing therapy in an asynchronous therapy mode or a synchronous therapy mode.

The pre-programmed period of time, which may also be referred to as a pre-determined CSR therapy duration time period, may include a specific amount of time that electrical stimulation is provided to a patient. It may be desirable to provide electrical stimulation only until the patient is no longer exhibiting the CSR pattern and thus no longer needs electrical stimulation. For example, limiting stimulation in this manner may minimize energy consumption (and thus, for example, extend battery life), minimize disruption to a patient's daily activities, minimize patient discomfort, and may minimize diaphragm fatigue that may otherwise be caused by pushing the diaphragm harder with stimulation rather than natural breathing.

In some examples, however, patient respiration may appear to be normal while electrical stimulation is delivered at act 606 because of the ongoing electrical stimulation, but may revert to exhibiting a CSR pattern as soon as stimulation is discontinued. Thus, even if the patient's respiration is or appears normal while electrical stimulation is delivered at act 606, it may not be apparent if the patient's respiration is normal because the CSR event has ended or only because electrical stimulation is being provided and the patient's respiration will again exhibit the CSR pattern if electrical stimulation is discontinued. It may therefore be advantageous to provide electrical stimulation for the pre-programmed period of time (for example, an expected duration of a CSR event) at act 606, reduce the electrical stimulation energy, and re-analyze the patient respiration to redetermine if the patient respiration again exhibits the CSR pattern.

At act 608, after the pre-programmed time period of act 606 elapses, the processor 50 controls the stimulation circuitry 42 to reduce the electrical stimulation energy. The processor 50 (for example, the non-sleep-period mode controller 76) controls the stimulation circuitry 42 to reduce the electrical stimulation energy from an initial value to a reduced value at the conclusion of the pre-programmed time period of act 606. In some examples, the reduced value is zero, that is, the electrical stimulation is discontinued completely. In these examples, the processor 50 may discontinue providing the electrical stimulation at the conclusion of the pre-programmed time period of act 606. In other examples, the reduced value is greater than zero but less than the initial value, that is, the electrical stimulation is still provided but with a reduced intensity.

At act 610, the processor 50 monitors patient respiration by monitoring one or more respiration parameters after reducing the electrical stimulation energy at act 608. As discussed in greater detail below, the processor 50 may execute the respiration monitor 74 to monitor patient respiration. Monitoring the one or more respiration parameters may include monitoring a patient-respiration signal from at least one respiration sensor. Act 610 may be substantially similar or identical to act 602.

At act 612, the processor 50 analyzes the monitored patient-respiration signal to determine whether the CSR pattern detected at act 604—YES is still present. As discussed in greater detail below, the processor 50 may execute the CSR identification engine 78 to determine whether the CSR pattern is still present. Act 612 may include executing a CSR-pattern detection algorithm similar to those discussed above with respect to act 604. In various examples, the processor 50 may execute the same detection algorithm or a different detection algorithm at acts 604 and 612. In some examples, the processor 50 may execute the same type of detection algorithm (for example, an amplitude-variation analysis, a cross-correlation analysis, a frequency-domain analysis, and so forth) at acts 604 and 612 but with different parameters. For example, the processor 50 may execute an amplitude-variation analysis at both acts 604 and 612; however, the threshold amplitude variation implemented to determine whether a CSR pattern is identified or not identified may differ between acts 604 and 612. For example, in some examples, the processor 50 may implement more sensitive CSR-detection conditions (such as a lower threshold amplitude variation) at act 612.

The processor 50 may repeatedly analyze the monitored patient-respiration signal until the processor 50 either detects a CSR pattern (in which case the processor 50 determines that the CSR pattern is still present [612 YES]) or a minimum amount of time has elapsed since reducing the electrical stimulation energy at act 608 without detecting a CSR pattern. The minimum amount of time may be, for example, a pre-determined period of time or a pre-determined number of breathing cycles of the monitored respiration. For example, the minimum amount of time may be 30 seconds, or one minute, or five minutes, or 10 minutes, or two breathing cycles, or 10 breathing cycles, or some other amount of time.

If the processor 50 determines based on the respiration signal monitored at act 610 and analyzed at act 612 that the CSR pattern is no longer present (612 NO), then the process 600 continues to act 614.

At act 614, the processor 50 discontinues the electrical stimulation. For example, the processor 50 may execute the non-sleep-period mode controller 76 to control the stimulation circuitry 42 to stop delivering electrical stimulation (which may simply include discontinuing controlling the stimulation circuitry 42 to deliver electrical stimulation). The process 600 then returns to act 602.

Otherwise, if the processor 50 redetermines based on the respiration signal monitored at act 610 and analyzed at act 612 that the CSR pattern is still present (612 YES) after the reduction in the electrical stimulation energy, then the process 600 may optionally continue to act 616. In some examples, optional act 616 may be omitted, and the process 600 may instead continue from act 612—YES to act 606.

In examples in which the processor 50 executes optional act 616, at act 616, the processor 50 adjusts at least one electrical stimulation parameter of the electrical stimulation responsive to the determination of the presence of the CSR pattern. For example, the processor 50 may execute the non-sleep-period mode controller 76 to adjust the electrical stimulation, such as by adjusting the electrical stimulation parameters used to control the stimulation circuitry 42. Because the CSR pattern is still present (612 YES) despite the electrical stimulation being provided to the patient at act 606, it may be advantageous to increase the intensity of the electrical stimulation. Increasing the intensity of the electrical stimulation may increase the efficacy of the CSR therapy. As discussed in greater detail below, the processor 50 may increase the intensity of the electrical stimulation by adjusting one or more stimulation parameters, such as by increasing an amplitude of the electrical stimulation, increasing the duration of the electrical stimulation, decreasing an amount of time between stimulation pulses, and so forth. In various examples, the processor 50 may increase the energy of the electrical stimulation in one or more steps to a maximum energy level (or until it is no longer necessary to increase the energy of the electrical stimulation) over one or more successive deliveries of the electrical stimulation while the CSR pattern persists. In other examples, the processor 50 may increase the energy of the electrical stimulation in one or more steps to a maximum energy level over a pre-programmed time period for a single electrical stimulation delivery. The processor 50 may increase the electrical stimulation from an initial energy level up to a maximum energy level over one or more of successive electrical stimulation delivery time periods or within a single electrical stimulation delivery time period. For example, the single electrical stimulation delivery time period may be the pre-programmed time period. The successive electrical stimulation delivery time periods may be successive stimulation deliveries for durations corresponding to the pre-programmed time period.

In some examples, act 616 may include adjusting the electrical stimulation based on a patient fluid load. The processor 50 may monitor a signal from at least one patient fluid load sensor coupled to the IPG 12 (the signal being indicative of a fluid load of the patient) and may adjust an energy of the electrical stimulation (or another electrical stimulation parameter) based on the signal from the at least one patient fluid load sensor. For example, the fluid load sensor may include the TTI sensors 32. The fluid load of the patient may refer to an amount of fluid in a patient's body. Some patients, such as those diagnosed with heart failure or renal disease, may be more likely to retain fluid than other patients. A fluid load may affect the efficacy of electrical stimulation provided to the patient. Accordingly, the processor 50 may adjust the electrical stimulation based on the patient's fluid load, such as by increasing the energy of the electrical stimulation for patients with a higher fluid load.

The process 600 then continues to act 606. Act 606 is then repeated by providing a second delivery of the electrical stimulation with the same electrical stimulation parameters as a preceding iteration of act 606 (for example, if the processor 50 does not execute optional act 616) or with different electrical stimulation parameters (for example, if the processor 50 executes optional act 616). In various examples, the processor 50 may implement the same pre-programmed delivery time period as a preceding iteration of act 606 or may implement a different pre-programmed delivery time period.

As discussed above, at act 606 the processor 50 delivers electrical stimulation for a pre-programmed time period. The processor 50 may implement acts 608-612 to determine whether the CSR pattern is absent from patient respiration when stimulation is no longer provided. As discussed above, the monitored respiration may not exhibit a CSR pattern while stimulation is provided, but may resume exhibiting the CSR pattern after stimulation is reduced or discontinued.

In some examples, however, the monitored respiration may exhibit CSR patterns even while electrical stimulation is provided at act 606. Accordingly, as discussed above and in FIG. 6B below, act 606 may include adjusting the electrical stimulation to treat the CSR effectively such that the monitored respiration does not exhibit CSR.

FIG. 6B illustrates a process 650 of providing electrical stimulation according to an example. The process 650 may be an example of act 606, or may be an example of acts that the processor 50 performs simultaneously while executing act 606. The processor 50 may execute the process 650 repeatedly while executing act 606, that is, while delivering electrical stimulation to a patient.

At act 651, the processor 50 determines whether to deliver asynchronous stimulation or whether to instead deliver synchronous stimulation. As noted above, the system 10 may be configured to change between modes automatically based on programming and/or other settings and/or may change between modes in response to instructions from the external computing device 210 and/or the patient-specific external computing device 215. If the processor 50 determines that the system 10 will deliver asynchronous stimulation (651 YES), then the process 650 continues to act 652 to deliver electrical stimulation to a patient's phrenic nerve for a pre-programmed time period.

At act 652, the processor 50 monitors a respiration signal indicative of one or more patient respiration parameters from at least one respiration sensor. The processor 50 may execute the respiration monitor 74 to monitor the respiration signal while providing electrical stimulation to the patient. The monitoring performed at act 652 may be substantially similar or identical to that performed in acts 602 and 610 (although in some examples the processor 50 may not be providing electrical stimulation during execution of acts 602 and 610).

At act 654, the processor 50 determines whether a CSR pattern is present in the respiration signal monitored at act 652. The processor 50 may execute the CSR identification engine 78 to determine whether the CSR pattern is present. If the CSR pattern is present even while stimulation is provided to the patient, the processor 50 may determine that the electrical stimulation should be adjusted. Act 654 may be substantially similar or identical to acts 604 and 612 discussed above. In some examples, however the processor 50 may execute the same type of detection algorithm (for example, an amplitude-variation analysis, a cross-correlation analysis, a frequency-domain analysis, and so forth) at act 654 as compared to acts 604 and 612 but with different parameters. For example, the processor 50 may execute an amplitude-variation analysis at both acts 604 and 654; however, the threshold amplitude variation implemented to determine whether a CSR pattern is identified or not identified may differ between acts 604 and 654. For example, in some examples, the processor 50 may implement more sensitive CSR-detection conditions (such as a lower threshold amplitude variation) at act 654. In other examples, the processor 50 may execute the same detection algorithm at act 654 as in other acts.

If the processor 50 determines that the CSR pattern is not present in the respiration signal (654 NO), then the process 650 returns to act 652. The processor 50 may continue to deliver electrical stimulation with the same stimulation parameters and may continue to monitor the respiration signal. The processor 50 repeats acts 652 and 654 until the pre-determined stimulation time period expires or the processor 50 determines based on the respiration signal that the CSR pattern is present (654 YES), in response to which the process 650 continues to act 656.

At act 656, the processor 50 adjusts one or more electrical stimulation parameters. In various examples, act 656 may be substantially similar or identical to act 616, and may include the processor 50 executing the non-sleep-period mode controller 76 to adjust parameters of the electrical stimulation discussed below with respect to FIG. 8. The process 650 then returns to act 652. The processor 50 may repeat acts 652, 654, and in some examples act 656 (for example, if the CSR pattern is present [654 YES]) throughout the pre-programmed time period. As discussed above with respect to FIG. 6A, when the pre-programmed time period of act 606 expires (and thus the processor 50 stops executing the process 650), the processor 50 may execute act 608.

Returning to act 651, if the processor 50 determines that the system 10 is to provide synchronous stimulation (651 NO), then the process 650 continues to act 658 to deliver synchronous stimulation, optionally with an apnea-triggered stimulation feature, to a patient's phrenic nerve for a pre-programmed time period.

At act 658, the processor 50 monitors a respiration signal indicative of one or more patient respiration parameters from at least one respiration sensor. Act 658 may be substantially similar or identical to act 652, and a description thereof will not be repeated for brevity.

At act 660, the processor 50 determines whether a CSR pattern is present in the respiration signal monitored at act 658. Act 660 may be substantially similar or identical to act 654, and a complete description thereof will not be repeated for brevity. Similar to act 654, if the processor 50 determines that the CSR pattern is not present in the respiration signal (660 NO), then the process 650 returns to act 658. The processor 50 may continue to deliver electrical stimulation with the same stimulation parameters and may continue to monitor the respiration signal. The processor 50 repeats acts 658 and 660 until the pre-determined stimulation time period expires or the processor 50 determines based on the respiration signal that the CSR pattern is present (660 YES), in response to which the process 650 continues to act 662.

At act 662, the processor 50 adjusts one or more electrical stimulation parameters. Act 662 may be substantially similar or identical to act 656, and a description thereof will not be repeated for brevity. The process 650 then returns to act 658.

Accordingly, FIG. 6A provides an example in which electrical stimulation may be provided (either asynchronously or synchronously) based on detecting a CSR pattern at act 604. In other examples, the system 10 may deliver stimulation based not only on detecting a CSR pattern, but also based on detecting a CSR precursor condition. That is, the system 10 may deliver stimulation if a CSR precursor condition is detected, or if a CSR pattern is detected, or a combination thereof. A CSR precursor condition is a condition that may indicate that a CSR event is likely to occur in the future but has not yet occurred. As discussed in regard to FIG. 6C, these precursor conditions may include but are not limited to one or more of patient motion, patient respiration amplitude, patient respiration rate, and/or patient tidal volume exceeding or subceeding a threshold or being within or outside of a target range.

Detecting a CSR precursor condition may be particularly advantageous for a waking patient as compared to a sleeping patient. For example, suppose that a patient (either waking or sleeping) is experiencing disordered breathing despite stimulation being provided. For a waking patient, a competition between stimulated breathing and the patient's CSR-disordered breathing may cause noticeable discomfort. For a sleeping patient, however, the patient may not be disturbed or awoken by this competition. Accordingly, for a waking patient, it may be more advantageous to provide stimulation in response to a CSR precursor condition such that stimulation is provided to address a CSR event before it occurs, thereby avoiding a situation in which a waking patient is subjected to the competition between a CSR event and stimulated breathing. In some examples, the system may require detection of both a CSR precursor and the CSR event in order to provide therapy. In such examples, the detection of the CSR precursor may cause the system to relax the CSR detection conditions. For example, the system may collect respiration data for a shorter period of time than in the absence of the CSR precursor and/or may provide a lower similarity threshold between respiration pattern cycles in order to identify the presence of CSR.

FIG. 6C illustrates a process 601 of providing waking CSR therapy according to another example. The process 601 is similar to the process 600, but in the process 601, the processor 50 monitors for at least one CSR precursor condition. The process 601 may include acts which are similar or identical to those of the process 600, and these acts are labeled accordingly. The processor 50 may execute the process 601.

At act 602, the processor 50 monitors patient respiration by monitoring one or more respiration parameters. Act 602 of the process 601 is substantially similar or identical to act 602 of the process 600 and a description thereof will not be repeated for brevity.

At act 603, the processor 50 monitors for one or more CSR-related conditions. For example, the processor 50 may monitor one or more of various CSR precursor parameters, such as one or more of patient motion, patient respiration amplitude, patient respiration rate, and/or patient tidal volume, or may monitor for an apnea. Act 603 may include the processor 50 determining whether the CSR-related parameters satisfy one or more corresponding CSR related conditions. For example, the processor 50 may determine one or more of whether the patient motion falls below a resting threshold corresponding to the patient being at rest, and/or may determine whether the patient respiration amplitude remains below a threshold value for a threshold amount of time (for example, three to 10 seconds) corresponding to an apnea, and/or may determine whether the patient respiration rate decreases below a threshold rate (for example, a fixed rate such as 12 breaths-per-minute or a relative amount such as 10-50% from a baseline respiration rate), and/or may determine whether a tidal volume falls below a threshold value (for example, a 30-50% decrease in tidal volume as indicated by a transthoracic impedance measurement). In other examples, other CSR precursor conditions may be monitored for.

In various examples, a patient may be experiencing an apnea when the patient's respiration remains below a threshold value for at least a threshold period of time. For example, a patient may be experiencing an apnea if the amplitude of the patient's respiration does not exceed a threshold value within, say, 3-10 seconds. As similarly discussed above in regard to FIG. 5A, upon detection of the apnea as the CSR-related condition, the system 10 may provide the stimulation according to an apnea-triggered asynchronous stimulation mode.

If the processor 50 determines that the CSR-related conditions are satisfied (603 YES), then the process 601 continues to act 606. As discussed above, act 606 may include the system 10 delivering electrical stimulation. Accordingly, the system 10 may deliver electrical stimulation responsive to detecting the CSR-related conditions without necessarily detecting the presence of a CSR pattern.

Conversely, if the processor 50 does not detect that the CSR-related conditions are satisfied (603 NO), then the process 601 continues to act 604. As discussed above, act 604 may include the processor 50 determining whether the CSR pattern is present and, if so (604 YES), delivering electrical stimulation at act 606.

Similar to the process 600, the processor 50 may then reduce the electrical stimulation energy at act 608 and monitor patient respiration during the reduced stimulation energy at act 610. The processor 50 then determines whether one or more CSR-related conditions are satisfied at act 611. The one or more CSR-related conditions evaluated at act 611 may be the same or different than those evaluated at act 603. If the processor 50 determines that the one or more CSR-related conditions are satisfied (611 YES), then the process 601 continues to act 616. Otherwise, if the processor 50 determines that the one or more CSR-related conditions are not satisfied (611 NO), then the process 601 continues to act 613.

At act 613, the processor 50 determines whether a CSR pattern is detected. Act 613 differs from act 612 of the process 600 inasmuch as a CSR pattern has not necessarily been previously detected in the process 601; for example, the processor 50 may execute act 613 after determining that the one or more CSR precursor conditions are satisfied at act 603—YES, such that the processor 50 may not have executed act 604. Act 613 may otherwise be substantially similar or identical to act 604 if a CSR pattern has not previously been detected, and may be substantially similar or identical to act 612 if a CSR pattern has previously been detected.

If the processor 50 determines that the CSR pattern is not detected (613 NO), then the process 601 continues to act 614. Otherwise, if the processor 50 determines that the CSR pattern is detected (613 YES), then the process 601 continues to optional act 616 or, if optional act 616 is not executed, to act 606. Acts 614 and 616 are substantially similar to those of the process 600, and a description thereof will not be repeated.

Accordingly, in some examples, the processor 50 may monitor for a CSR precursor condition and may take certain actions (for example, delivering electrical stimulation at act 606) if a CSR precursor condition is detected, whether or not a CSR pattern is present or detected. That is, the processor 50 may execute act 606 if either acts 603—YES or 604—YES are satisfied. In other examples, the processor 50 may monitor for a CSR precursor condition, but may only take certain actions (for example, delivering electrical stimulation at act 606) if both a CSR precursor condition is satisfied and a CSR pattern is detected. That is, the processor 50 may execute act 606 only if both acts 603—YES and 604—YES are satisfied. In some examples, the detection of the CSR precursor may cause the system to relax the CSR detection conditions. For example, the system may collect respiration data for a shorter period of time than in the absence of the CSR precursor and/or may provide a lower similarity threshold between respiration pattern cycles in order to identify the presence of CSR.

In some examples, the processor 50 may execute the processes 600, 601, 650 and/or variations thereof while the current time is within a pre-determined non-sleep time period. However, as mentioned above and discussed in more detail here, in contrast to stimulation therapy delivered to a sleeping patient, in some examples, the processor 50 may terminate or temporarily suspend executing the processes 600, 601, 650 and/or monitoring for CSR and delivering CSR therapy responsive to one or more criteria being met that indicate a likelihood that detected periodic breathing may be attributable to activities of an awake patient. During stimulation therapy to a sleeping patient and/or for a device that only delivers stimulation therapy when patient conditions indicate that the patient is asleep, the device and the stimulation therapy need not account for periodic breathing due to patient activities of the awake patient. These activities may include exercising, activity, talking, eating, etc.

For example, it may be necessary to pause stimulation therapy while a patient is moving or exercising, because such therapy may interfere with necessary patient control of breathing. In some examples, therefore, the processor 50 may temporarily suspend delivering CSR therapy responsive to determining that patient movement exceeds a threshold. The processor 50 may monitor a signal indicative of patient motion from at least one patient motion sensor, such as the accelerometer 38, to determine whether the patient movement exceeds the patient movement threshold. For example, the patient movement threshold may include a minimum speed or acceleration. If the processor 50 determines that the patient movement exceeds a patient movement threshold, then the processor 50 may suspend any ongoing electrical stimulation and/or refrain from initiating any electrical stimulation. In some examples, the processor 50 may resume providing electrical stimulation after, for example, a minimum period of time elapses or after the patient's breathing is no longer heavily influenced by exercising.

In another example, the processor 50 may terminate or temporarily suspend providing stimulation therapy (such as by discontinuing electrical stimulation or initiating a period during which no stimulation is provided) responsive to receiving a first user input to stop or pause electrical stimulation. For example, the processor 50 may receive the first user input via the wireless communications interface 52. The patient may provide the first user input to the wireless communications interface 52 via the patient-specific external computing device 215. The patient may terminate or pause the electrical stimulation because they are about to eat, or exercise, or engage in some other activity during which electrical stimulation will interfere with necessary patient control of breathing. For example, stimulation that causes a person to inhale while eating or drinking may result in an adverse aspiration of food or water and possible choking.

In some examples, the processor 50 may pause electrical stimulation for a period of time until at least one resumption criterion is met. In one example, a resumption criterion may include the patient providing, via the patient-specific external computing device 215 to the wireless communications interface 52, a second user input to resume the electrical stimulation. In another example, a resumption criterion may include the processor 50 determining that a pre-determined period of time has elapsed since receiving the first user input. The processor 50 may resume providing the electrical stimulation responsive to the at least one resumption criterion being met.

In various examples, the IPG 12 may provide an alert to the patient via the patient-specific external computing device 215 indicative of a resumption or an initiation of the electrical stimulation responsive to one or more resumption criteria being met. For example, the IPG 12 may automatically commence delivery of the electrical stimulation a pre-determined amount of time after providing the alert. In another example, the IPG 12 may not commence delivery of the electrical stimulation until the patient provides a third user input in response to the alert, the third user input indicating that the IPG 12 may resume providing electrical stimulation.

In at least one example, the IPG 12 may provide an alert to the patient-specific external computing device 215 indicative of an initiation of the electrical stimulation prior to delivery of the electrical stimulation in other circumstance, such as each time the processor 50 enters the non-sleep time therapy mode of operation (for example, at the beginning of the non-sleep time). In another example, the processor 50 may enter the non-sleep time therapy operational mode during the pre-determined non-sleep time period for the patient responsive to a user input therapy commencement instruction provided to the IPG 12 by the patient-specific external computing device 215 (for example, via the wireless communications interface 52).

Accordingly, in some examples the processor 50 (individually or in concert with other components, such as the stimulation circuitry 42) may execute various acts of the processes 500, 600, 601, 650 to deliver waking CSR therapy and/or sleep apnea therapy. In other examples, one or more acts of the processes 500, 600, 601, 650 may be executed at least in part by the processor 50 and at least in part by other components, such as the external computing devices 210 and/or 215. For example, as discussed above, the processor 50 may monitor a respiration signal and may derive information therefrom. Although in some examples the processor 50 may execute a CSR detection algorithm at act 604 based on the respiration signal or the information derived therefrom, in other examples, the processor 50 may provide the respiration signal and/or information derived therefrom to one or both of the external computing devices 210 and/or 215. One or both of the external computing devices 210 and/or 215 may implement and execute the CSR detection algorithm using the respiration signal and/or the information derived therefrom. The processor 50 may receive a CSR pattern detection indication from at least one of the external computing devices 210 and/or 215, such that the processor 50 making a determination of the presence of the CSR pattern at act 604 is based on the CSR pattern detection indication received from at least one of the external computing devices 210 and/or 215.

Accordingly, the system 10 may deliver stimulation therapy to a patient during a waking period (arbitrarily referred to as “daytime” stimulation) and during a sleeping period (arbitrarily referred to as a “nighttime” period). For convenience of explanation, a patient is assumed to be awake during the daytime, and is assumed to be asleep during the nighttime in the following discussion. However, as discussed above, patients may sleep during the day and be awake during the night.

The system 10 may deliver waking stimulation and sleeping stimulation may according to various stimulation modes. The stimulation mode during the waking period may be the same as or different than the stimulation mode during the sleeping period. Furthermore, the system 10 may change the stimulation mode corresponding to either or both of the waking and sleeping periods during operation.

As discussed above, the stimulation modes may include a synchronous stimulation mode, an asynchronous stimulation mode, and an apnea-triggered asynchronous stimulation mode. Daytime stimulation therapy may include any of these three stimulation modes, and the system 10 may change between these stimulation modes throughout the daytime stimulation therapy. Similarly, nighttime stimulation therapy may include any of these three stimulation modes, and the system 10 may change between these stimulation modes throughout the nighttime stimulation therapy. The daytime stimulation therapy may be the same mode, or may be a different mode, than the nighttime stimulation mode.

For example, in one example, the daytime stimulation mode may be the synchronous stimulation mode and the nighttime stimulation mode may be the synchronous stimulation mode. In another example, the daytime stimulation mode may be the synchronous stimulation mode and the nighttime stimulation mode may be the asynchronous stimulation mode. In another example, the daytime stimulation mode may be the synchronous stimulation mode and the nighttime stimulation mode may be the apnea-triggered asynchronous stimulation mode.

In another example, the daytime stimulation mode may be the asynchronous stimulation mode and the nighttime stimulation mode may be the synchronous stimulation mode. In another example, the daytime stimulation mode may be the asynchronous stimulation mode and the nighttime stimulation mode may be the asynchronous stimulation mode. In another example, the daytime stimulation mode may be the asynchronous stimulation mode and the nighttime stimulation mode may be the apnea-triggered asynchronous stimulation mode.

In another example, the daytime stimulation mode may be the apnea-triggered asynchronous stimulation mode and the nighttime stimulation mode may be the synchronous stimulation mode. In another example, the daytime stimulation mode may be the apnea-triggered asynchronous stimulation mode and the nighttime stimulation mode may be the asynchronous stimulation mode. In another example, the daytime stimulation mode may be the apnea-triggered asynchronous stimulation mode and the nighttime stimulation mode may be the apnea-triggered asynchronous stimulation mode. As noted above, the system 10 may transition between any of these combinations of stimulation modes over time.

Stimulation may be provided in accordance with stimulation parameters, such as current amplitude, pulse width, and so forth. Stimulation parameters may remain constant or may change over time. Furthermore, the stimulation parameters of daytime stimulation may be the same as or different than the stimulation parameters of nighttime stimulation. As discussed with respect to FIG. 6D, various scenarios may be supported in which different combinations of stimulation parameters are provided for daytime and nighttime stimulation.

FIG. 6D illustrates a table 670 depicting various scenarios of providing stimulation to a waking and sleeping patient. In the illustrated example, the table 670 includes three example scenarios (Scenario 1 through Scenario 3). Each of the scenarios includes at least one stimulation mode for a waking patient and at least one stimulation mode for a sleeping patient. For convenience of explanation, a patient is assumed to be awake during the “Daytime” in FIG. 6D, and is assumed to be asleep during the “Nighttime” in FIG. 6D, as indicated by the therapy regime column 682. However, as discussed above, patients may sleep during the day and be awake during the night. Each patient may have a different pre-determined sleep therapy period which may or may not correspond to sleeping during the night and being awake during the day. Accordingly, while a patient is assumed for ease of explanation to have a pre-determined sleep therapy period that corresponds to the “Nighttime,” no limitation is implied.

Stimulation parameters 686 are provided for each stimulation scenario. One stimulation parameter includes stimulation rate. For convenience of explanation, the table 670 provides scenarios in which stimulation is provided on an asynchronous basis, that is, the stimulation rate is pre-determined and the system 10 thus provides stimulation in the asynchronous stimulation mode and/or the apnea-triggered asynchronous stimulation mode. Stimulation provided for the sleeping patient may therefore correspond to act 514, and stimulation provided for the waking patient may correspond to act 506. The principles of FIG. 6D may also be applicable to the synchronous stimulation mode; however, the stimulation rate may be determined based on the patient's respiration rate rather than being a pre-determined value. Accordingly, the scenarios outlined in FIG. 6D are not identified as corresponding to a particular stimulation type because the principles of the stimulation parameters 686 (with the exception of stimulation rate) may be applicable to any of the stimulation modes.

The parameters 686 provided in FIG. 6D are examples only and not limiting of the disclosure. These values serve to illustrate the ability and capability of the system 10 to provide stimulation according to different parameters depending on the regime 682.

For a sleeping patient in Scenario 1, the amplitude of the current of the electrical stimulation may vary between a minimum value of 3 mA and a maximum value of 3.8 mA. The pulse width of the electrical stimulation (for example, the pulse width 860) may be approximately 150 usec. The frequency of the electrical stimulation pulses may be approximately 20 Hz. The rate of electrical stimulation pulse trains may be approximately 12 pulse trains per minute. A duty cycle of the inspiration-to-expiration ratio (I:E) may be approximately 1.

Each pulse train may ramp up according to a 30/10/50 ramping scheme. As discussed below with respect to the ramping-up pulses 898 and ramping-down pulses 899, a pulse train may ramp up from a baseline value to a constant-amplitude value, hold the amplitude at the constant amplitude, and then ramp down. In the 30/10/50 ramping scheme, the first number (“30”) indicates that the ramping-up pulses 898 ramp up for 30% of the pulse train duration 850 before reaching the constant-amplitude value. The second number (“10”) indicates that the ramping-down pulses 899 ramp down for 10% of the pulse train duration 850 before the electrical stimulation drops to zero. Thus, for 60% of the pulse train duration 850 (that is, 100% 30%-10%), the amplitude of the pulse train is held constant. The third number (“50”) indicates that the initial amplitude of the pulse train (that is, the value that the ramping-up pulses 898 ramp up from) starts at 50% of a maximum stimulation amplitude.

For a waking patient in Scenario 1, the amplitude of the electrical stimulation current varies between 2.8 mA and 3.5 mA, and the pulse width, frequency, and I:E are substantial similar or identical to those of the nighttime stimulation. However, the rate of delivery may be higher (for example, approximately 14 pulse trains per minute) and the ramping scheme may be 30/30/50, that is, while the ramping-up duration and baseline may be the same as during the nighttime, the ramping-down duration (30%) may be longer than in the sleeping therapy (10%).

Accordingly, as discussed above and as illustrated by Scenario 1, the electrical stimulation may be less intense during the daytime than during the nighttime. A patient who is awake may be more aware of the discomfort caused by electrical stimulation than if the patient were asleep. Providing less-intense electrical stimulation during the day may therefore increase patient comfort while the patient is awake without compromising therapy efficacy while the patient is asleep.

Thus, as illustrated by Scenario 1, stimulation intensity may be decreased while a patient is awake. If the electrical stimulation nonetheless causes patient discomfort, the nighttime and/or daytime stimulation may be decreased. Alternatively, if the electrical stimulation is not sufficiently intense to be as effective as desired, the nighttime and/or daytime stimulation may be increased. For example, Scenario 2 indicates an example in which the same types of therapy are provided, but in which the stimulation parameters have been modified.

In Scenario 2, the stimulation parameters are modified between nighttime and daytime therapy even where both therapies are delivered in an asynchronous therapy mode and are modified as compared with Scenario 1. For example, during the nighttime, the stimulation amplitude varies between 5 mA and 6.5 mA, the pulse width is approximately 300 usec, the frequency is approximately 40 Hz, the rate is approximately 18 pulse trains per minute, the I:E is 1, and the ramping scheme is 30/10/50. During the daytime, the stimulation amplitude varies between 4.5 mA and 6 mA, the I:E ratio is approximately 1.5, and the ramping scheme is 20/20/50. The pulse width, frequency, and rate are approximately the same as during the nighttime stimulation.

In addition to Scenario 2 enhancing patient comfort compared to Scenario 1, shortening the ramping-up and ramping-down durations and increasing the I:E compared to Scenario 1 may create longer peak inhalation hold times to encourage more air intake by a patient. Accordingly, as illustrated by Scenarios 1 and 2, the same types of therapy modes may be provided in response to the same conditions in different scenarios, but the stimulation parameters dictating the properties of the stimulation provided to the patient may be adjusted based on the needs of a patient.

In Scenario 3, during the daytime, the stimulation amplitude may vary between 1.5 mA and 1.9 mA, the pulse width may be approximately 60 usec, the frequency may be approximately 40 Hz, the rate may be approximately 10 pulse trains per minute, the I:E may be approximately 1.0, and the ramping scheme may follow a 30/10/50 scheme. During the nighttime for Scenario 3, the stimulation parameters 686 may be substantially similar or identical to the daytime, except that the stimulation rate may be increased to increase patient comfort. For example, the rate may be approximately 12 pulse trains per minute.

Accordingly, the scenarios of FIG. 6D illustrate examples of how stimulation parameters 686 may differ between nighttime stimulation and daytime stimulation. Although FIG. 6D illustrates examples of asynchronous stimulation for convenience of explanation, other stimulation parameters 686 may also be varied in synchronous stimulation modes. Furthermore, although stimulation parameters 686 are illustrated for various scenarios of daytime and nighttime stimulation, the stimulation parameters 686 may vary throughout a daytime period and a nighttime period, that is, the stimulation parameters 686 may not be fixed.

As discussed above, daytime stimulation may be provided in response to various conditions, such as a time-based condition and either or both of a CSR pattern or a CSR precursor condition being detected. Similarly, nighttime stimulation may be provided in response to various conditions, such as a time-based condition and motion-based conditions. FIG. 6D is agnostic of these various conditions, that is, FIG. 6D may be applicable to daytime and nighttime stimulation modes regardless of what conditions must be met to activate the stimulation. In various examples, the stimulation parameters 686 may therefore not be dictated by the conditions that trigger stimulation.

Various acts of the processes 500, 600, 601, 650 may be executed at least in part by the stimulation mode controller 72, the respiration monitor 74, the non-sleep-period mode controller 76, the CSR identification engine 78, the sleep apnea mode controller 80 (including the RDS evaluator 88), the timer 82, the clock 84, and/or the sleep condition evaluator 86. In various examples, the processor 50 may be configured to be able to execute firmware and/or software to select stimulation modes to enter or exit, as described below in regard to FIGS. 7A-7D.

For example, FIG. 7A illustrates a swim-lane diagram 700 depicting operation of the processor 50 according to an example. The example of FIG. 7A may correspond to a situation in which the time is within a pre-determined sleep time period (504 YES) and the sleep indication conditions are satisfied (510 YES). Accordingly, the example of FIG. 7A may correspond to a same or similar example as an execution of the process 500 in which acts 502, 504, and 508, 510, 512, and 514 and/or 516 are executed. According to the example of FIGS. 7A-7D, the system 100 may be able to provide stimulation therapy for an awake patient and for a sleeping patient. However, these are examples only and not limiting of the disclosure. In an implementation, the system 100 may only provide stimulation therapy for the awake patient and may not be able to provide stimulation therapy for the sleeping patient based on settings from software, firmware, and/or hardware.

At act 702, the clock 82 provides time information to the sleep condition evaluator 86. The sleep condition evaluator 86 evaluates the time information to determine that the current time is within a pre-determined sleeping time period. For example, if a patient has a pre-determined sleeping time period of between 8 PM on Monday to 7 AM on Tuesday, and the current time indicates that the time is 10 PM on Monday, then the current time is within the pre-determined sleeping time period.

At act 704, the sleep condition evaluator 86 evaluates other sleep indication conditions. The sleep condition evaluator 86 may evaluate one or more a signal indicative of motion and/or position parameters provided by at least one patient motion sensor, such as the accelerometer 38, to determine whether the parameters satisfy the sleep indication conditions. For example, the sleep condition evaluator 86 may determine based on one or more motion parameters whether a degree of patient movement exceeds a patient movement threshold. The sleep condition evaluator 86 may determine based on one or more position parameters whether the patient is in one of a list of patient sleeping positions, such as lying on one's back, stomach, or side, as opposed to non-sleeping positions, such as sitting up or standing. In various examples, the sleep condition evaluator 86 may perform an evaluation based on either or both of the motion or position parameters.

At act 706, the sleep condition evaluator 86 determines that the sleep indication conditions are met. For example, the sleep condition evaluator 86 may determine that the patient movement is below the patient movement threshold and/or that the patient position is one of a pre-determined list of sleeping positions. The sleep condition evaluator 86 provides an indication of the sleep indication conditions being met to the stimulation mode controller 72.

At act 708, the stimulation mode controller 72 selects a stimulation mode to enter. For example, stimulation modes may include a waking CSR stimulation mode or a sleep apnea stimulation mode. Because the sleep condition evaluator 86 indicates to the stimulation mode controller 72 that the patient is likely asleep, the stimulation mode controller 72 selects a sleep apnea stimulation mode and provides an indication thereof to the sleep apnea mode controller 80.

At act 710, the sleep apnea mode controller 80 triggers sleep-apnea-therapy electrical stimulation by controlling the stimulation circuitry 42 to provide stimulation. Controlling the stimulation circuitry 42 to provide stimulation may include selecting one or more electrical stimulation parameters for the sleep-apnea-therapy electrical stimulation, examples of which are discussed below with respect to FIG. 8. In some examples, the sleep apnea mode controller 80 may trigger stimulation based on information received from the respiration monitor 74. For example, the sleep apnea mode controller 80 may trigger stimulation responsive to determining, based on a respiration signal from the respiration monitor 74, that the patient's respiration exhibits a sleep-apnea pattern, such as OSA or CSA.

At act 712, the stimulation circuitry 42 provides sleep-apnea-therapy electrical stimulation to a patient. For example, the stimulation circuitry 42 may draw power from the energy source 54 and provide electrical stimulation based on the one or more stimulation parameters selected by the non-sleep-period mode controller 76. The stimulation circuitry 42 may provide the electrical stimulation to the patient via one or more of the leads 14, 16.

Accordingly, in some examples, the processor 50 may provide sleep-apnea stimulation therapy to a patient during a sleeping time period in which motion and/or position parameters indicative of patient movement are indicative of a patient being asleep. In other examples, the processor 50 may not provide stimulation therapy if, during the sleeping time period, the motion and/or position parameters are not indicative of a patient being asleep.

For example, FIG. 7B illustrates a swim-lane diagram 720 depicting operation of the processor 50 according to an example. The example of FIG. 7B may correspond to a situation in which the time is within a pre-determined sleep time period (504 YES), but in which the sleep indication conditions are not satisfied (510 NO). Accordingly, the example of FIG. 7B may correspond to a same or similar example as an execution of the process 500 in which acts 502, 504, 508, and 510 are executed, but not acts 506, 514, or 516.

At act 722, the clock 82 provides time information to the sleep condition evaluator 86. The sleep condition evaluator 86 evaluates the time information to determine that the current time is within a pre-determined sleeping time period. For example, if a patient has a pre-determined sleeping time period of between 8 PM on Monday to 7 AM on Tuesday, and the current time indicates that the time is 10 PM on Monday, then the current time is within the pre-determined sleeping time period.

At act 724, the sleep condition evaluator 86 evaluates other sleep indication conditions. Similar to act 704, the sleep condition evaluator 86 may evaluate one or more a signal indicative of motion and/or position parameters provided by at least one patient motion sensor, such as the accelerometer 38, to determine whether the parameters satisfy the sleep indication conditions.

At act 726, the sleep condition evaluator 86 determines that the sleep indication conditions are not met. For example, the sleep condition evaluator 86 may determine that the patient movement is above the patient movement threshold and/or that the patient position is one of a pre-determined list of non-sleeping positions. In the illustrated example, the sleep condition evaluator 86 provides an indication of the sleep indication conditions not being met to the stimulation mode controller 72. In other examples, the sleep condition evaluator 86 may take no action in response to the sleep indication conditions not being met.

At act 728, the stimulation mode controller 72 does not select a stimulation mode and thus provides no stimulation to a patient. For example, because the processor 50 determines that the current time is not within a pre-determined non-sleeping time period, the processor 50 does not provide any waking CSR therapy. However, because the processor 50 determines that the patient movement is indicative of the patient being awake still, the processor 50 does not provide any sleep apnea therapy.

At act 730, the sleep condition evaluator 86 may repeatedly re-evaluate sleep indication conditions, including a current time and patient movement parameters. As discussed above with respect to FIG. 7A, the processor 50 may provide sleep-apnea stimulation therapy if the sleep condition evaluator 86 later determines that the sleep indication conditions are met.

Accordingly, in some examples, the processor 50 may provide no stimulation therapy to a patient during a sleeping time period in which motion and/or position parameters indicative of patient movement are indicative of a patient being awake still, despite the current time being within the sleeping time period. In other examples, the processor 50 may provide waking CSR therapy to a patient during a non-sleeping time period.

For example, FIG. 7C illustrates a swim-lane diagram 740 depicting operation of the processor 50 according to another example. The example of FIG. 7C may correspond to a situation in which the time is within a pre-determined non-sleep time period (504 NO). Accordingly, the example of FIG. 7C may correspond to a same or similar example as an execution of the process 500 in which act 506 is executed along with the process 600.

At act 742, the clock 82 provides time information to the sleep condition evaluator 86. The sleep condition evaluator 86 evaluates the time information to determine that the current time is within a pre-determined non-sleep time period. For example, if a patient has a pre-determined non-sleeping time period of between 7 AM to 8 PM on Monday, and the current time indicates that the time is 5 PM on Monday, then the current time is within the pre-determined non-sleeping time period.

At act 744, the sleep condition evaluator 86 determines that the one or more sleep indication conditions are not met because the current time is outside of the pre-determined sleep time period. As discussed above, an example sleep indication condition may include the current time being within a pre-determined sleep time period, that is, outside of a pre-determined non-sleep time period. Because the current time is outside of the pre-determined sleep time period and within a pre-determined non-sleep time period, the sleep condition evaluator 86 determines that the one or more sleep indication conditions are not met and provides information indicative of the current time being within the pre-determined non-sleep time period to the stimulation mode controller 72.

At act 746, the stimulation mode controller 72 selects the non-sleep time period stimulation mode of operation. The stimulation mode controller 72 may activate the respiration monitor 74 to begin monitoring patient respiration to detect one or more CSR patterns. In some examples, the stimulation mode controller 72 may also provide a signal to the non-sleep-period mode controller to enter the non-sleep time therapy operational mode and/or may activate the CSR identification engine 78 to analyze a monitored respiration signal to determine whether a CSR pattern is present in the monitored respiration signal.

At act 748, the respiration monitor 74 monitors a respiration signal from one or more respiration sensors. The respiration monitor 74 provides information indicative of the monitored respiration to the CSR identification engine 78.

At act 750, the CSR identification engine 78 determines based on the respiration signal that the CSR pattern is present. As discussed above, the CSR identification engine 78 may execute one or more detection algorithms to determine whether a CSR pattern is present in the respiration signal, such as a cross-correlation analysis, an amplitude-variation analysis, and/or a FFT analysis. In some examples, the CSR identification engine 78 may record a CSR marker in the memory 56 indicative of the detection of the CSR pattern. The CSR identification engine 78 may provide a notification of the identified CSR pattern to the non-sleep time mode controller 76.

At a therapy delivery stage 752, which may be repeatedly executed, various aspects of the IPG 12 may operate in concert to provide waking CSR therapy to a patient. In some examples, the therapy delivery stage 752 may be repeatedly executed until the CSR pattern is no longer present in the respiration signal. The therapy delivery stage 752 may include acts 754-766, one or more of which may be optionally executed or omitted in various examples.

At act 754, the non-sleep period mode controller 76 triggers electrical-stimulation delivery by the stimulation circuitry 42. Triggering the electrical stimulation may include selecting one or more electrical stimulation parameters of the electrical stimulation and controlling the stimulation circuitry 42 accordingly. For example, the processor 50 may cause the stimulation circuitry 42 to deliver the electrical stimulation to the leads 13 by controlling one or more switching elements to modulate power (for example, derived from the power supply 54) and produce an electrical stimulation with the selected electrical stimulation parameters.

At act 756, the stimulation circuitry 42 provides electrical stimulation to the patient. The electrical stimulation may be CSR therapy stimulation delivered to a patient who is awake and who is experiencing a waking CSR event. The stimulation circuitry 42 may provide the electrical stimulation for at least a pre-determined and pre-programmed time period which is timed by the timer 84.

At act 758, the timer 84 begins timing a pre-determined stimulation time period responsive to the simulation circuitry 42 beginning to provide electrical stimulation. As discussed above, the processor 50 may provide electrical stimulation for a pre-determined amount of time, reduce the electrical stimulation, and re-evaluate whether a CSR pattern is present based on respiration signals received during the period of reducing the electrical stimulation. The timer 84 may provide an indication of how much time has elapsed to the non-sleep period mode controller 76 while timing the pre-determined stimulation time period.

At act 760, the respiration monitor 74 monitors patient respiration. Act 760 may be substantially similar or identical to act 748. The respiration monitor 74 provides information indicative of the monitored patient respiration to the CSR identification engine 78. As discussed above with respect to FIG. 6B, the processor 50 may monitor patient respiration for a CSR pattern even while electrical stimulation is provided to the patient.

At optional act 762, the CSR identification engine 78 may optionally detect a CSR pattern in the monitored respiration signal while the stimulation circuitry 42 delivers electrical stimulation to the patient. In other examples, the CSR identification engine 78 may not detect any CSR patterns in the monitored respiration signal. For purposes of example, however, if the CSR identification engine 78 does detect one or more CSR patterns, the CSR identification engine 78 provides an indication thereof to the non-sleep time period mode controller 76.

At optional act 764, which may be optionally executed if the CSR identification engine 78 detects a CSR pattern, the non-sleep time period mode controller 76 adjusts the electrical stimulation. As discussed above with respect to acts 654 and 656, if the CSR identification engine 78 detects a CSR pattern in the respiration signal even while waking CSR therapy is provided to the user, the electrical stimulation may not be intense enough to effectively treat the CSR event. Accordingly, the non-sleep time period mode controller 76 may adjust the electrical stimulation by increasing the intensity of the electrical stimulation to more effectively treat the CSR event. Adjusting the electrical stimulation may include modifying one or more electrical stimulation parameters. In other examples, optional 764 may not be executed, such as if the CSR identification engine 78 does not detect any CSR patterns in the respiration signal.

In some examples, acts 760-764 may be repeatedly executed until the pre-determined stimulation time period elapses.

At act 766, the non-sleep time period mode controller 76 reduces the electrical stimulation responsive to the pre-determined stimulation time period elapsing. Reducing the electrical stimulation may include the non-sleep time period mode controller 76 modifying one or more electrical stimulation parameters that are provided to, or govern the control of, the stimulation circuitry 42. For example, the non-sleep time period mode controller 76 may reduce an amplitude of the electrical stimulation from an initial value to a final value, with the final value being less than the initial value. In some examples, the final value is zero, that is, the non-sleep time period mode controller 76 discontinues providing electrical stimulation. In other examples, the final value is less than the initial value but greater than zero, that is, the non-sleep time period mode controller 76 continues controlling the stimulation circuitry 42 to provide electrical stimulation, but with a reduced amplitude.

In some examples, the CSR identification engine 78 may again determine whether one or more CSR patterns are present in the respiration signal while the reduced electrical stimulation is provided to the patient. If the CSR identification engine 78 again detects that a CSR pattern is present, as discussed above with respect to act 750, the treatment delivery stage 752 may be repeated. Repeating the treatment delivery stage 752 may include, for example, the non-sleep period mode controller 76 again triggering the stimulation circuitry 42 to provide electrical stimulation (as discussed with respect to act 754) with an increased intensity as compared to the reduced electrical stimulation at act 766. In some examples, the processor 50 may repeat the treatment delivery stage 752 until the CSR identification engine 78 determines that a CSR pattern is not present.

At act 770, the CSR identification engine 78 determines that a CSR pattern is not detected in the respiration signal. As discussed above, in some examples, the CSR identification engine 78 may analyze the respiration signal for a minimum period of time after the non-sleep period mode controller 76 reduces the electrical stimulation. For example, the CSR identification engine 78 may analyze the respiration signal for a pre-determined minimum amount of time or for a pre-determined number of breaths. The CSR identification engine 78 provides an indication that the CSR pattern is no longer present to the non-sleep time period mode controller 76 responsive to determining that a CSR pattern is not present.

At act 772, the non-sleep time period mode controller 76 discontinues controlling the stimulation circuitry 42 to provide stimulation to a patient. The system 10 therefore stops providing waking CSR therapy to a patient because, for example, the patient's respiration no longer exhibits a CSR pattern.

As a further example, FIG. 7D illustrates a swim-lane diagram 741 depicting operation of the processor 50 according to another example. The example of FIG. 7D may correspond to a situation in which the time is within a pre-determined non-sleep time period (504 NO). Accordingly, the example of FIG. 7D may correspond to a same or similar example as an execution of the process 500 in which act 506 is executed along with the process 601. FIG. 7D is substantially similar to FIG. 7C with the exception of the added act 749. Thus, the discussion of 7C will not be repeated here for brevity.

At act 749, the CSR identification engine 78 determines based on the respiration signal that a CSR precursor condition is present. For example, the CSR identification engine 78 may determine one or more of whether the patient motion falls below a resting threshold corresponding to the patient being at rest, and/or may determine whether the patient respiration amplitude remains below a threshold value for a threshold amount of time (for example, three to 10 seconds) corresponding to an apnea, and/or may determine whether the patient respiration rate decreases below a threshold rate (for example, a fixed rate such as 12 breaths-per-minute or a relative amount such as 10-50% from a baseline respiration rate), and/or may determine whether a tidal volume falls below a threshold value (for example, a 30-50% decrease in tidal volume as indicated by a transthoracic impedance measurement). In other examples, other CSR precursor conditions may be monitored for.

Optionally, the CSR identification engine 78 performs the act 749 in conjunction with the act 750 to identify the presence of the CSR pattern. The act 750 is similarly described in regard to FIG. 7C. In some examples, the detection of the CSR precursor may cause the CSR identification engine 78 to relax the CSR detection conditions. For example, the system may collect respiration data for a shorter period of time than in the absence of the CSR precursor and/or may provide a lower similarity threshold between respiration pattern cycles in order to identify the presence of CSR. In some examples, the CSR identification engine 78 may record a CSR marker and/or a CSR precursor marker in the memory 56 indicative of the detection of the CSR pattern and/or the CSR precursor. The CSR identification engine 78 may provide a notification of the identified CSR pattern and/or the CSR precursor to the non-sleep time mode controller 76.

As discussed above, modifying the electrical stimulation may include adjusting one or more electrical stimulation parameters. FIG. 8 illustrates an example of an electrical stimulation trace 890 depicting various electrical stimulation parameters. The electrical stimulation includes a series of stimulation pulses 880 or stimulation bursts 880. Each stimulation pulse 880 may be provided as a pulse train 891 where the stimulation pulse 880 is a pulse envelope for the individual pulses (for example, the n pulses 885a, 885b, . . . , 885n) of the pulse train 891. Each stimulation pulse 880 may be referred to as a stimulation burst 880 as the pulse train 891 provides a burst of individual pulses. The leads 14, 16 may each include electrodes to deliver electrical stimulation to a target nerve according to the electrical stimulation parameters the processor 50 (for example, the non-sleep-period mode controller 76 and/or the sleep apnea mode controller 80) selects for the electrical stimulation.

The stimulation pulses 880 are delivered at a stimulation pulse period corresponding to a stimulation rate. The stimulation rate may be about 0.1-0.5 Hz in correspondence with a desired stimulated breathing rate of 10-30 breaths per minute. In some examples, a stimulation rate (for example, indicated by a stimulation profile of the stimulation profile library 57) may be determined based on a predetermined unstimulated respiration rate, that is, a predetermined respiration rate when stimulation is not applied. For example, the stimulation rate may be equal to the predetermined unstimulated respiration rate, or may be greater or less than the predetermined unstimulated respiration rate. In one example, the stimulation rate may be a predetermined number of cycles-per-minute fewer than the predetermined unstimulated respiration rate (for example, one cycle-per-minute fewer, two cycles-per-minute fewer, three cycles-per-minute fewer, or some other value). In other examples, the stimulation rate is a predetermined number of cycles-per-minute greater than the predetermined unstimulated respiration rate (for example, one cycle-per-minute greater, two cycles-per-minute greater, three cycles-per-minute greater, or some other value).

Each stimulation pulse 880 may be about one to four seconds in total duration. In the example of FIG. 8, the total duration of the stimulation pulse 880 is the pulse train duration 850. For example, the stimulation pulse may be about two seconds in total duration. Each stimulation pulse 880 may be provided as a pulse train having a pulse train period 866 corresponding to a pulse train frequency. The pulse train frequency (that is, the frequency of the individual pulses within the stimulation pulse 880 or stimulation burst 880) may be about 10-40 Hz.

The controller 50 may select electrical stimulation parameters including one or more of the stimulation rate (corresponding to the stimulation pulse period 865), the pulse train duration 850, and the pulse train frequency (corresponding to the pulse train period 866). Additionally, the parameters may include a time between stimulation pulses 830. The ratio of the stimulation pulse width (or duration) 850 to the stimulation pulse period 865 defines the stimulation pulse duty cycle. The pulse train envelope forming each stimulation pulse 880 defines the maximum pulse amplitude 840 as the maximum amplitude of one or more individual pulses within the pulse train. Each individual pulse may be further characterized by a pulse width 860. As illustrated in FIG. 8, the individual pulses of the pulse train may not all have the same pulse amplitude 845 and may vary in amplitude so as to ramp up 898 the energy of the stimulation pulse 880 and/or ramp down 899 the energy of the stimulation pulse 880. The ramp up may occur over a ramping-up time, for example, the time interval for the ramping up pulses 898. The ramp down may occur over a ramping-down time, for example, the time interval for the ramping down pulses 899. In some examples, the stimulation pulse 880 may not include a ramp up and/or a ramp down. The IPG 12 configuration with regard to operational parameters and/or with regard to stimulation circuitry 42 may exclude a square wave capability for an electrical stimulation profile in which the pulse trains exclude both the ramping-up period and the ramping-down period.

As discussed above, the controller 50 may adjust one or more of the electrical stimulation parameters to titrate the electrical stimulation energy delivered to a target nerve. The electrical stimulation parameters may include pulse train parameters. The pulse train parameters, as discussed above, may include one or more of a pulse amplitude 845, a maximum pulse amplitude 840, a pulse train period (or frequency) 866, a pulse width 860, and/or a pulse train duration 850. The pulse amplitude 845 may include an amplitude of at least one of the ramping-up pulses, an amplitude of the constant-amplitude pulses, and/or an amplitude of at least one of the ramping-down pulses. The pulse train parameters may further include one or more of a time over which an amplitude of the pulses is ramped up, a time over which the amplitude of the pulses is ramped down, a number of ramping-up pulses, and/or a number of ramping-down pulses.

The stimulation parameters may further include one or more of the stimulation rate corresponding to the stimulation pulse period 865, the time between stimulation pulses 830, a stimulation current, a stimulation voltage, and a stimulation polarity (for example, monophasic or biphasic), stimulation energy ramps, and so forth. The stimulation energy ramps may include changes in energy delivered by a stimulation pulse 880 over a series of stimulation pulses 880. One or more electrical stimulation parameters may be adjusted to change the energy delivered by stimulation pulses 880 over time to titrate the stimulation energy delivered to the target nerve. Such titration may achieve a desirable balance between patient comfort and stimulation efficacy. For example, more intense electrical stimulation of a patient's phrenic nerve may yield higher activation of a patient's diaphragm, which may increase the efficacy of nerve stimulation therapy in some examples. Each stimulation profile 58 may also include titration parameters for titration of stimulation energy over a period of minutes, hours, days, months, and so forth.

However, more intense electrical stimulation may also yield a greater sensory response from a patient. For example, a patient may feel more discomfort as the stimulation intensity is increased. Experiencing discomfort may adversely impact a patient, whether the patient is awake or asleep. For example, even if a particular set of stimulation parameters effectively activate the patient's diaphragm, treatment according to that particular set of stimulation parameters may be contraindicated if the particular set of stimulation parameters causes the patient to experience discomfort. It is therefore beneficial to the efficacy of stimulation therapy to identify stimulation parameters that produce a desired muscle response (for example, diaphragm contraction and expansion) while minimizing or avoiding patient discomfort. This may be true for various different treatment scenarios in which different combinations of therapy modes, each of which triggers therapy according to different conditions, are provided.

In an implementation, the IPG 12 may ramp up the stimulation amplitude to increase the intensity of the stimulation, or may ramp down the stimulation amplitude to decrease the intensity of the stimulation. In general, the stimulation amplitude may vary over a range from a minimum amplitude for a pulse train to a maximum amplitude for the pulse train. The individual pulses within the pulse train may vary in amplitude but the maximum amplitude defines the maximum amplitude reached during a stimulation pulse train. In at least one example, individual pulse train pulses that constitute a stimulation pulse may ramp up for a period of time and may ramp down for a period of time. In various examples, a start time of a pulse train may refer to a time at which a pulse train begins to ramp up, and an end time of a pulse train may refer to a time at which a pulse train finishes ramping down.

FIG. 9 illustrates a block diagram of a computing device 900 according to an example. The computing device 900 may illustrate an example of either or both of the computing devices 210, 215. While in some examples the computing device 900 is a tablet type of computer or a mobile phone such as a smartphone, the computing device 900 can include other types of computers and is therefore described in the context of a general computing device. In its most basic configuration, the computing device 900 includes at least a processing unit 102 and a memory 105. Depending on the exact configuration and type of computing device, the memory 105may be volatile (such as RAM), non-volatile (such as ROM, flash memory, and so forth) or some combination of the two. This most basic configuration is illustrated in FIG. 9 within box 107.

Additionally, the computing device 900 may also have additional features and/or functionality. For example, the computing device 900 may also include additional storage (removable and/or non-removable) including, but not limited to, magnetic or optical disks or tapes, USB flash drives, memory cards, and so forth. Such additional storage is illustrated in FIG. 9 by a removable storage 108 and a non-removable storage 110. Computer-storage media may include volatile and/or nonvolatile media, removable and/or non-removable media, and so forth, implemented in a method or technology for storage of information such as computer-readable instructions, data structures, program modules or other data. The memory 105, the removable storage 108, and the non-removable storage 110 are all examples of computer-storage media, also referred to as non-transitory computer-readable media. Computer-storage media includes, but is not limited to, RAM, ROM, EEPROM, flash memory or other memory technology, CDROM, digital versatile disks (DVD) or other optical storage, magnetic cassettes, magnetic tape, magnetic disk storage or other magnetic storage devices, or another medium that can be used to store the desired information and which can be accessed by the computing device 900. Any such computer-storage media may be part of computing device 900.

System memory 105 may include operating system 130, one or more programming modules or applications 132, and program data 134. Operating system 130, for example, may be suitable for controlling the operation of the computing device 900. As stated above, a number of program modules 132 and program data files 134 may be stored in system memory 105, including operating system 130. While executing on processing unit 102, programming modules or applications 132 may perform processes including, for example, one or more methods described herein, using one or more of the GUI screens or windows shown and described herein. For example, where the computing device 900 is an example of the external computing device 210 and/or the patient-specific external computing device 215, the processing unit 102 may include and/or execute the RDS evaluator 26.

The program modules or applications 132 may include one or more of routines, programs, components, data structures, and other types of structures that may perform particular tasks or that may implement particular abstract data types. Moreover, disclosed examples may be practiced with other computer-system configurations, including multiprocessor systems, microprocessor-based or programmable consumer electronics, minicomputers, mainframe computers, and the like. Disclosed examples may also be practiced in distributed computing environments where tasks are performed by remote processing devices linked through a communications network. In a distributed computing environment, program modules may be located in both local and remote memory storage devices.

Furthermore, some disclosed examples may be practiced in an electrical circuit including discrete electronic elements, packaged or integrated electronic chips containing logic gates, a circuit utilizing a microprocessor, or on a single chip containing electronic elements or microprocessors. Some disclosed examples may also be practiced using other technologies capable of performing logical operations such as, for example, AND, OR, and NOT, including but not limited to mechanical, optical, fluidic, and quantum technologies.

The computing device 900 may also contain a communications interface 112 that allows the device to communicate with other devices. The communications interface 112 can include, for example, wired media connections such as a wired network or direct-wired connection, and wireless media connections such as acoustic, RF, infrared, and other wireless media connections. In some examples, communications interface 112 is configured to provide communication (for example, wireless communication) via a connection 902 between the computing device 900 and the IPG 12 of treatment system 10. For example, such communications may include processor instructions provided by a programmer of the IPG 12 to select certain stimulation profiles or parameters thereof. Thus, in some examples, the communication interface 112 may include circuitry configured to provide communication connection as a wireless communication connection as described above.

In still other examples, such as in which the computing device 900 is coupled with or replaced by remote or cloud-based computing device(s) 38 for some or all of the processing functions described herein, communication interface 112 can communicate via a connection 904 to an internet connection or other network 37, to the computing device(s) 38, which is communicatively coupled to the network 37 via a connection 906. In still other examples, the processing functions described herein are performed without the use of a local computing device 900, and are instead incorporated into the treatment system 10 and/or the computing device(s) 38. In some examples, the treatment system 10 performs at least a portion of the processing functions described herein, and a remaining portion, if any, of the processing functions may be performed by the computing device 900 and/or one or more alternative or additional computing devices, such as the remote or cloud-based computing devices 38. The description herein is provided in the context of processing functions being provided at least partially by computing device 900, but those of skill in the art will understand that such functions can be implemented outside of computing device 900.

In some examples the computing device 900 has, or can be coupled to, a touch screen display device 116 which provides a touch-based GUI. The computing device 900 may also have, or be coupled to, one or more input devices 114, such as a keyboard, mouse, pen, voice input device, and so forth, for providing other input (for example, user feedback) to the computing device 900. The computing device 900 may be coupled to one or more other output devices 118 such as speakers, a printer, a vibration generator, and so forth. Further, display device 116, input devices 114 and output devices 118 can all be considered to be separate from, or alternatively part of, the computing device 900. The computing device 900 can be provided with a portable or non-portable power source 120, such as a battery pack, a transformer, a power supply, or the like. The power source 120 provides power for computations, communications and so forth by the computing device 900.

As discussed above, in certain examples a sleeping time period and a non-sleeping time period may be set for a patient. The periods may collectively make up a 24-hour period. In some examples, only an indication of a sleep time period or a non-sleep time period may be pre-determined. However, because the sleep and non-sleep time periods may be complementary with one another over a 24-hour period, determining whether a current time is within a sleep or non-sleep time period may inherently include determining whether the current time is not within a non-sleep time period or a sleep time period. That is, because a pre-determined sleep time period may be used to determine that the complementary time periods over a 24-hour period are non-sleep periods, and vice versa, storing an indication of one pre-determined time period may effectively be used to determine the other time period. Accordingly, in various examples, an indication of a sleep time period may be pre-determined, or an indication of a non-sleep time period may be pre-determined, or an indication of both a sleep and non-sleep time period may be pre-determined.

Various controllers, such as the processor 50, may execute various operations discussed above. The processor 50 may be or include one or more hardware components and may be or include processing circuitry. The processor 50may also execute one or more instructions stored on one or more non-transitory computer-readable media, which the processor 50may include and/or be coupled to, which may result in manipulated data. The one or more non-transitory computer-readable media may be or include hardware devices. The non-transitory computer-readable media may include memory and/or storage hardware. In some examples, the processor 50 may include one or more processors or other types of controllers. Example processors may include hardware components such as microprocessors. In another example, the processor 50 performs at least a portion of the operations discussed above using an application-specific integrated circuit tailored to perform particular operations in addition to, or in lieu of, a processor. As illustrated by these examples, examples in accordance with the present disclosure may perform the operations described herein using many specific combinations of hardware and software and the disclosure is not limited to any particular combination of hardware and software components. Examples of the disclosure may include a computer-program product configured to execute methods, processes, and/or operations discussed above. The computer-program product may be, or include, one or more controllers and/or processors configured to execute instructions to perform methods, processes, and/or operations discussed above. The computer-program product may be, or include, at least one hardware component configured to store and/or execute at least one computer program, and may be or include processing circuitry.

Having thus described several aspects of at least one embodiment, it is to be appreciated various alterations, modifications, and improvements will readily occur to those skilled in the art. Such alterations, modifications, and improvements are intended to be part of, and within the spirit and scope of, this disclosure. Accordingly, the foregoing description and drawings are by way of example only.

What is claimed is:

1. An implantable pulse generator (IPG) for treating Cheyne-Stokes respiration (CSR) in an awake patient, the IPG configured to provide electrical stimulation to a target tissue comprising a phrenic nerve via at least one implantable stimulation lead configured to couple to the IPG, the IPG comprising:
at least one processor, at least one memory, and stimulation circuitry; and
wherein the at least one processor is configured to
enter a non-sleep time therapy operational mode during a pre-determined non-sleep time period for a patient,
monitor a signal from at least one respiration sensor coupled to the IPG,
identify CSR of the patient based at least in part on a determination of a presence of a CSR pattern in the monitored signal, and
control the stimulation circuitry to
provide a first delivery of the electrical stimulation to the phrenic nerve for a pre-programmed time period in response to the determination of the presence of the CSR pattern in the monitored signal.

2. The IPG of claim 1, wherein the at least one processor is configured to
control the stimulation circuitry to reduce an energy of the electrical stimulation at a conclusion of the pre-programmed time period,
redetermine the presence of the CSR pattern in the monitored signal after the reduction in the electrical stimulation energy, and
if the redetermination indicates the presence of the CSR pattern in the monitored signal, then control the stimulation circuitry to provide a second delivery of the electrical stimulation to the phrenic nerve for the pre-programmed time period, else discontinue the electrical stimulation.

3-5. (canceled)

6. The IPG of claim 1, wherein the at least one processor is configured to discontinue delivery of the electrical stimulation at a conclusion of a pre-determined CSR therapy duration time period.

7. The IPG of claim 1, wherein the at least one processor is further configured to monitor a signal from at least one patient motion sensor, and discontinue the electrical stimulation responsive to the signal from the at least one patient motion sensor indicative of patient movement above a patient movement threshold.

8. The IPG of claim 1, comprising a wireless communications interface configured to communicate with an external computing device via one or more of telemetry or a Bluetooth® communications protocol.

9. The IPG of claim 8, wherein the at least one processor is further configured to
store a CSR event marker in the at least one memory, and
transmit the CSR event marker to the external computing device via the wireless communications interface.

10-12. (canceled)

13. The IPG of claim 8, wherein the at least one processor is further configured to discontinue the electrical stimulation responsive to reception, via the wireless communications interface, of a first user input to the external computing device to stop the electrical stimulation.

14. The IPG of claim 13, wherein the at least one processor is further configured to automatically resume providing the electrical stimulation after a pre-determined period of time elapses after reception of the first user input to stop the electrical stimulation or resume providing the electrical stimulation responsive to reception of a second user input to the external computing device to resume the electrical stimulation.

15. (canceled)

16. The IPG of claim 8, wherein the IPG is further configured to provide an alert to the external computing device indicative of an initiation of the electrical stimulation prior to delivery of the electrical stimulation.

17. The IPG of claim 16, wherein the at least one processor is further configured to automatically commence delivery of the electrical stimulation a pre-determined amount of time after providing the alert.

18. (canceled)

19. The IPG of claim 8, wherein the IPG is further configured to output an alarm indicative of the CSR pattern determination to the external computing device.

20. (canceled)

21. The IPG of claim 1, wherein the identification of CSR of the patient comprises implementation of a detection algorithm to determine the presence of the CSR pattern in the monitored signal for at least a threshold duration.

22. The IPG of claim 21, wherein the at least one processor is configured to implement the detection algorithm.

23. The IPG of claim 21, wherein the IPG is configured to
communicatively couple to at least one external computing device configured to implement the detection algorithm,
provide at least one of the monitored signal or information derived from the signal from the monitored signal to the at least one external computing device, and
receive a CSR pattern detection indication from the at least one external computing device,
wherein the determination of the presence of the CSR pattern in the monitored signal is based on the CSR pattern detection indication.

24. (canceled)

25. The IPG of claim 21, wherein the threshold duration comprises one or more complete cycles of the CSR pattern, at least two complete cycles of the CSR pattern, or 30-120 seconds.

26. (canceled)

27. The IPG of claim 21, wherein the detection algorithm is further configured to determine a correlation index between signal morphology elements of the monitored signal and identify the signal morphology elements as CSR cycles based on the correlation index.

28. The IPG of claim 21,
wherein the at least one processor is configured to receive a sensor signal indicative of patient activity from one or more of an implanted accelerometer or an accelerometer coupled to an exterior surface of the patient, and
wherein the determination of the presence of the CSR pattern in the monitored signal is based at least in part on the patient activity.

29. (canceled)

30. The IPG of claim 1, wherein the at least one processor is configured to increase an energy of the electrical stimulation from an initial energy level up to a maximum energy level over the pre-programmed time period or over one or more successive stimulation delivery time periods.

31. The IPG of claim 1,
wherein the at least one processor is configured to enter the non-sleep time therapy operational mode based at least in part on a pre-programmed start time-of-day and a pre-programmed end time-of-day,
wherein the at least one processor is further configured to enter a sleep apnea therapy operational mode during a pre-determined sleep time period for the patient identified at least in part based on the pre-programmed start time-of-day and the pre-programmed end time-of-day, and
wherein the at least one processor is further configured to control the stimulation circuitry to deliver electrical stimulation during the pre-determined sleep time period at a higher energy than during the pre-determined non-sleep time period.

32-44. (canceled)

45. The IPG of claim 1, wherein the at least one respiration sensor comprises a transthoracic-impedance sensor.

46-110. (canceled)
